(19) United States
(12) Patent Application Publication
(10) Pub. No.: US 2025/0223356 A1
(43) Pub. Date: Jul. 10, 2025

(54) METHODS AND COMPOSITIONS FOR TREATING RELAPSING MULTIPLE SCLEROSIS

(71) Applicant: Zenas BioPharma, Inc., Waltham, MA (US)

(72) Inventors: Maria Melanson, Jacksonville, FL (US); Tanya Fischer, Newton, MA (US); Stephanie Ciarlone, Lorton, VA (US)

(21) Appl. No.: 19/013,625

(22) Filed: Jan. 8, 2025

Related U.S. Application Data
(60) Provisional application No. 63/618,762, filed on Jan. 8, 2024.
(60) Provisional application No. 63/724,759, filed on Nov. 25, 2024.

(51) Int. Cl. C07K 16/28; A61K 39/00

(52) U.S. Cl. CPC C07K 16/2803; A61K 2039/545; C07K 2317/52; C07K 2317/565; C07K 2317/76

(57) ABSTRACT

The present invention provides methods of treating and improving the symptoms of forms of multiple sclerosis using an antibody or antigen-binding fragment thereof that specifically binds human CD19 (e.g., obexelimab).

### CROSS-REFERENCE TO RELATED APPLICATIONS

This application claims the benefit of and priority to U.S. Application No. 63/618,762, filed Jan. 8, 2024, and U.S. Application No. 63/724,759, filed Nov. 25, 2024, the content of each of which are hereby incorporated by reference in their entireties.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Jan. 6, 2025, is named “ZEN-025US1_SL.xml” and is 13,212 bytes in size.

### BACKGROUND

Multiple sclerosis (MS) is a chronic autoimmune disease of the central nervous system (CNS). While the exact cause and mechanisms of the disease are unclear, MS is characterized by inflammation, demyelination, and neurodegeneration along with the formation of plaques or lesions within the CNS. Symptoms of MS are often varied and difficult to predict, but individuals with MS may experience pain, fatigue, cognitive difficulties, depression, mobility restrictions, lack of coordination, difficulties with speech or swallowing, and various vision disorders, as well as many other symptoms. Relapsing Multiple Sclerosis (RMS) is the most common form of MS, affecting 85% of MS patients. While disease-modifying therapies have been approved for the treatment of RMS, there is currently no known cure for RMS.

Current anti-CD20-based therapies for MS, including RMS, indiscriminately deplete nearly all B cells (Graf et al., 2021; Lee, D. S. W., et al., 2021. Nat. Rev. Drug Discov. 20:179-199; and Margoni, M., et al., 2022. J. Neurol. 269:1316-1334). People living with RMS on long-term treatment could conceivably go for decades missing much of a major branch of the immune system that is necessary for effective and protective immune responses. The long-term health outcomes of maintaining B cell depletion for much of a person's lifespan is not known but could leave individuals less able to mount protective responses to vaccines and increase susceptibility to infection. Further, when the choice is made for the individual to come off anti-CD20 therapy, return to normal immune function could potentially take months while B cells repopulate from the bone marrow (Margoni et al., 2022).

### SUMMARY OF INVENTION

The present invention provides, among other things, a method of treating forms of MS comprising administering to a patient in need of treatment an anti-CD19 antibody at a therapeutically effective dosing regimen. In some embodiments, an anti-CD19 antibody comprises a light chain and a heavy chain, wherein the light chain comprises a LCDR1 of SEQ ID NO:2, a LCDR2 of SEQ ID NO:3, and a LCDR3 of SEQ ID NO:4, and wherein the heavy chain comprises a HCDR1 of SEQ ID NO:5, a HCDR2 of SEQ ID NO:6, and a HCDR3 of SEQ ID NO:7, and wherein the heavy chain comprises an Fc region comprising amino acid substitutions S267E and L328F wherein numbering is according to the EU index. In some embodiments, an anti-CD19 antibody is obexelimab.

In some aspects, the present invention provides a method of treating relapsing forms of MS comprising administering to a patient in need of treatment an anti-CD19 antibody at a dose of 250 mg every week, or an equivalent dosing regimen thereof, wherein the anti-CD19 antibody comprises a light chain and a heavy chain, wherein the light chain comprises a LCDR1 of SEQ ID NO:2, a LCDR2 of SEQ ID NO:3, and a LCDR3 of SEQ ID NO:4, and wherein the heavy chain comprises a HCDR1 of SEQ ID NO:5, a HCDR2 of SEQ ID NO:6, and a HCDR3 of SEQ ID NO:7, and wherein the heavy chain comprises an Fc region comprising amino acid substitutions S267E and L328F wherein numbering is according to the EU index.

In some aspects, the present invention provides a method of treating primary-progressive multiple sclerosis (PPMS) comprising administering to a patient in need of treatment an anti-CD19 antibody at a dose of 250 mg every week, or an equivalent dosing regimen thereof, wherein the anti-CD19 antibody comprises a light chain and a heavy chain, wherein the light chain comprises a LCDR1 of SEQ ID NO:2, a LCDR2 of SEQ ID NO:3, and a LCDR3 of SEQ ID NO:4, and wherein the heavy chain comprises a HCDR1 of SEQ ID NO:5, a HCDR2 of SEQ ID NO:6, and a HCDR3 of SEQ ID NO:7, and wherein the heavy chain comprises an Fc region comprising amino acid substitutions S267E and L328F wherein numbering is according to the EU index.

In some embodiments, a therapeutically effective dosing regimen comprises administering an anti-CD19 antibody at a dose of 250 mg every week.

In some embodiments, a therapeutically effective dosing regimen comprises administering an anti-CD19 antibody at a dose of 250 mg every week or an equivalent dosing regimen thereof.

In some aspects, the present invention provides a method of treating MS comprising administering to a patient in need of treatment an anti-CD19 antibody at a dose of 250 mg every week, or an equivalent dosing regimen thereof, wherein the anti-CD19 antibody comprises a light chain and a heavy chain, wherein the light chain comprises a LCDR1 of SEQ ID NO:2, a LCDR2 of SEQ ID NO:3, and a LCDR3 of SEQ ID NO:4, and wherein the heavy chain comprises a HCDR1 of SEQ ID NO:5, a HCDR2 of SEQ ID NO:6, and a HCDR3 of SEQ ID NO:7, and wherein the heavy chain comprises an Fc region comprising amino acid substitutions S267E and L328F wherein numbering is according to the EU index.

In some embodiments, a patient has relapsing-remitting MS (RRMS), secondary progressive MS (SPMS), or clinically isolated syndrome (CIS). In some embodiments, a patient has RRMS. In some embodiments, a patient has SPMS. In some embodiments, a patient has CIS.

In some embodiments, a patient has an Expanded Disability Status Scale (EDSS) score of ≤5.5 prior to commencing treatment. In some embodiments, a patient has an EDSS score of ≤5.0 prior to commencing treatment. In some embodiments, a patient has an EDSS score of ≤4.5 prior to commencing treatment. In some embodiments, a patient has an EDSS score of ≤4.0 prior to commencing treatment. In some embodiments, a patient has an EDSS score of ≤3.5 prior to commencing treatment. In some embodiments, a patient has an EDSS score of ≤3.0 prior to commencing treatment. In some embodiments, a patient has an EDSS score of ≤2.5 prior to commencing treatment. In some embodiments, a patient has an EDSS score of ≤2.0 prior to commencing treatment. In some embodiments, a patient has an EDSS score of ≤1.5 prior to commencing treatment.

In some embodiments, a patient has an EDSS score of ≥5.0 prior to commencing treatment. In some embodiments, a patient has an EDSS score of ≥5.5 prior to commencing treatment. In some embodiments, a patient has an EDSS score of ≥6.0 prior to commencing treatment. In some embodiments, a patient has an EDSS score of ≥6.5 prior to commencing treatment. In some embodiments, a patient has an EDSS score of ≥7.0 prior to commencing treatment. In some embodiments, a patient has an EDSS score of ≥7.5 prior to commencing treatment. In some embodiments, a patient has an EDSS score of ≥8.0 prior to commencing treatment. In some embodiments, a patient has an EDSS score of ≥8.5 prior to commencing treatment. In some embodiments, a patient has an EDSS score of ≥9.0 prior to commencing treatment. In some embodiments, a patient has an EDSS score of ≥9.5 prior to commencing treatment.

In some embodiments, a patient has experienced at least one relapse within the previous year, or at least two relapses within the previous two years prior to commencing treatment. In some embodiments, a patient has experienced at least one relapse within the previous year prior to commencing treatment. In some embodiments, a patient has experienced at least two relapses within the previous two years prior to commencing treatment.

In some embodiments, administration of an anti-CD19 antibody is sufficient to stabilize or reduce EDDS score. In some embodiments, wherein the administration of an anti-CD19 antibody is sufficient to stabilize EDDS score. In some embodiments, wherein the administration of an anti-CD19 antibody is sufficient to reduce EDDS score.

In some embodiments, administration of an anti-CD19 antibody reduces the level of neurofilament light chain (NfL) in the patient's blood as compared to pre-treatment level.

In some embodiments, a patient has developed at least one active gadolinium-enhancing (GdE) brain lesion on a magnetic resonance imaging (MRI) scan within the past 6 months prior to commencing treatment. In some embodiments, a patient has developed at least one active GdE brain lesion on an MRI scan within the past 5 months prior to commencing treatment. In some embodiments, a patient has developed at least one active GdE brain lesion on an MRI scan within the past 4 months prior to commencing treatment. In some embodiments, a patient has developed at least one active GdE brain lesion on an MRI scan within the past 3 months prior to commencing treatment. In some embodiments, a patient has developed at least one active GdE brain lesion on an MRI scan within the past 2 months prior to commencing treatment. In some embodiments, a patient has developed at least one active GdE brain lesion on an MRI scan within the past 1 month prior to commencing treatment.

In some embodiments, administration of an anti-CD19 antibody is sufficient to stabilize or reduce GdE T1 hyperintense lesions as compared to pre-treatment status. In some embodiments, administration of an anti-CD19 antibody is sufficient to stabilize GdE T1 hyperintense lesions as compared to pre-treatment status. In some embodiments, administration of an anti-CD19 antibody is sufficient to reduce GdE T1 hyperintense lesions as compared to pre-treatment status.

In some embodiments, administration of an anti-CD19 antibody is sufficient to stabilizes or reduces GdE T2 hyperintense lesions as compared to pre-treatment status. In some embodiments, administration of an anti-CD19 antibody is sufficient to stabilizes GdE T2 hyperintense lesions as compared to pre-treatment status. In some embodiments, administration of an anti-CD19 antibody is sufficient to reduces GdE T2 hyperintense lesions as compared to pre-treatment status.

In some embodiments, an anti-CD19 antibody is administered for at least 2 weeks, 4 weeks, 8 weeks, at least 12 weeks, or at least 24 weeks. In some embodiments, an anti-CD19 antibody is administered for at least 2 weeks. In some embodiments, an anti-CD19 antibody is administered for at least 4 weeks. In some embodiments, an anti-CD19 antibody is administered for at least 6 weeks. In some embodiments, an anti-CD19 antibody is administered for at least 8 weeks. In some embodiments, an anti-CD19 antibody is administered for at least 10 weeks. In some embodiments, an anti-CD19 antibody is administered for at least 12 weeks. In some embodiments, an anti-CD19 antibody is administered for at least 14 weeks. In some embodiments, an anti-CD19 antibody is administered for at least 16 weeks. In some embodiments, an anti-CD19 antibody is administered for at least 18 weeks. In some embodiments, an anti-CD19 antibody is administered for at least 20 weeks. In some embodiments, an anti-CD19 antibody is administered for at least 22 weeks. In some embodiments, an anti-CD19 antibody is administered for at least 24 weeks. In some embodiments, an anti-CD19 antibody is administered for at least 36 weeks. In some embodiments, an anti-CD19 antibody is administered for at least 48 weeks. In some embodiments, an anti-CD19 antibody is administered for at least 52 weeks. In some embodiments, an anti-CD19 antibody is administered for at least 60 weeks. In some embodiments, an anti-CD19 antibody is administered for at least 72 weeks.

In some embodiments, a light chain comprises a light chain variable region that is at least 90% identical to SEQ ID NO:11, and a heavy chain comprises a heavy chain variable region that is at least 90% identical to SEQ ID NO:12.

In some embodiments, a light chain comprises a light chain variable region that is at least 90% identical to SEQ ID NO:11. In some embodiments, a light chain comprises a light chain variable region that is at least 91% identical to SEQ ID NO:11. In some embodiments, a light chain comprises a light chain variable region that is at least 92% identical to SEQ ID NO:11. In some embodiments, a light chain comprises a light chain variable region that is at least 93% identical to SEQ ID NO:11. In some embodiments, a light chain comprises a light chain variable region that is at least 94% identical to SEQ ID NO:11. In some embodiments, a light chain comprises a light chain variable region that is at least 95% identical to SEQ ID NO:11. In some embodiments, a light chain comprises a light chain variable region that is at least 96% identical to SEQ ID NO:11. In some embodiments, a light chain comprises a light chain variable region that is at least 97% identical to SEQ ID NO:11. In some embodiments, a light chain comprises a light chain variable region that is at least 98% identical to SEQ ID NO:11. In some embodiments, a light chain comprises a light chain variable region that is at least 99% identical to SEQ ID NO:11.

In some embodiments, a heavy chain comprises a light chain variable region that is at least 90% identical to SEQ ID NO:12. In some embodiments, a heavy chain comprises a heavy chain variable region that is at least 91% identical to SEQ ID NO:12. In some embodiments, a heavy chain comprises a heavy chain variable region that is at least 92% identical to SEQ ID NO:12. In some embodiments, a heavy chain comprises a heavy chain variable region that is at least 93% identical to SEQ ID NO:12. In some embodiments, a heavy chain comprises a heavy chain variable region that is at least 94% identical to SEQ ID NO:12. In some embodiments, a heavy chain comprises a heavy chain variable region that is at least 95% identical to SEQ ID NO:12. In some embodiments, a heavy chain comprises a heavy chain variable region that is at least 96% identical to SEQ ID NO:12. In some embodiments, a heavy chain comprises a heavy chain variable region that is at least 97% identical to SEQ ID NO:12. In some embodiments, a heavy chain comprises a heavy chain variable region that is at least 98% identical to SEQ ID NO:12. In some embodiments, a heavy chain comprises a heavy chain variable region that is at least 99% identical to SEQ ID NO:12.

In some embodiments, wherein a heavy chain variable region comprises SEQ ID NO:12; and a light chain variable region comprises SEQ ID NO:11.

In some embodiments, a heavy chain variable region comprises SEQ ID NO:12.

In some embodiments, a light chain variable region comprises SEQ ID NO:11.

In some embodiments, a heavy chain comprises an amino acid sequence at least 90% identical to SEQ ID NO:10, and a light chain comprises an amino acid sequence at least 90% identical to SEQ ID NO:9.

In some embodiments, a heavy chain comprises an amino acid sequence at least 90% identical to SEQ ID NO:10. In some embodiments, a heavy chain comprises an amino acid sequence at least 91% identical to SEQ ID NO:10. In some embodiments, a heavy chain comprises an amino acid sequence at least 92% identical to SEQ ID NO:10. In some embodiments, a heavy chain comprises an amino acid sequence at least 93% identical to SEQ ID NO:10. In some embodiments, a heavy chain comprises an amino acid sequence at least 94% identical to SEQ ID NO:10. In some embodiments, a heavy chain comprises an amino acid sequence at least 95% identical to SEQ ID NO:10. In some embodiments, a heavy chain comprises an amino acid sequence at least 96% identical to SEQ ID NO:10. In some embodiments, a heavy chain comprises an amino acid sequence at least 97% identical to SEQ ID NO:10. In some embodiments, a heavy chain comprises an amino acid sequence at least 98% identical to SEQ ID NO:10. In some embodiments, a heavy chain comprises an amino acid sequence at least 99% identical to SEQ ID NO:10.

In some embodiments, a light chain comprises an amino acid sequence at least 90% identical to SEQ ID NO:9. In some embodiments, a light chain comprises an amino acid sequence at least 91% identical to SEQ ID NO:9. In some embodiments, a light chain comprises an amino acid sequence at least 92% identical to SEQ ID NO:9. In some embodiments, a light chain comprises an amino acid sequence at least 93% identical to SEQ ID NO:9. In some embodiments, a light chain comprises an amino acid sequence at least 94% identical to SEQ ID NO:9. In some embodiments, a light chain comprises an amino acid sequence at least 95% identical to SEQ ID NO:9. In some embodiments, a light chain comprises an amino acid sequence at least 96% identical to SEQ ID NO:9. In some embodiments, a light chain comprises an amino acid sequence at least 97% identical to SEQ ID NO:9. In some embodiments, a light chain comprises an amino acid sequence at least 98% identical to SEQ ID NO:9. In some embodiments, a light chain comprises an amino acid sequence at least 99% identical to SEQ ID NO:9.

In some embodiments, a heavy chain comprises SEQ ID NO:10, and a light chain comprises SEQ ID NO:9.

In some embodiments, a heavy chain comprises SEQ ID NO:10.

In some embodiments, a light chain comprises SEQ ID NO:9.

In some embodiments, an anti-CD19 antibody is administered subcutaneously.

In some embodiments, an anti-CD19 antibody is administered in a liquid formulation comprising 125 mg/mL anti-CD19 antibody. In some embodiments, a liquid formulation further comprises 2.35 mg/mL sodium acetate trihydrate, 0.17 mg/mL acetic acid, 30 mg/mL L-proline, 0.1 mg/mL polysorbate 80 at pH 5.5.

In some embodiments, an anti-CD19 antibody is administered using a prefilled syringe.

In some embodiments, an anti-CD19 antibody is administered using an autoinjector.

### DETAILED DESCRIPTION

### Definitions

In order for the present disclosure to be more readily understood, certain terms are first defined below. Additional definitions for the following terms and other terms are set forth throughout the specification.

Described herein are several definitions. Such definitions are meant to encompass grammatical equivalents.

Antibody: The term “antibody” or other grammatical equivalents herein is meant to include a protein consisting of one or more polypeptides substantially encoded by all or part of the recognized immunoglobulin genes. The recognized immunoglobulin genes, for example in humans, include the kappa (K), lambda (l), and heavy chain genetic loci, which together comprise the myriad variable region genes, and the constant region gene gamma (y) which encodes the IgG (IgG1, IgG2, IgG3, and IgG4) isotype. Antibody herein is meant to include full length antibodies and antibody fragments, and may refer to a natural antibody from any organism, an engineered antibody, or an antibody generated recombinantly for experimental, therapeutic, or other purposes.

Effector Function: The term “effector function” or other grammatical equivalents as used herein is meant a biochemical event that results from the interaction of an antibody Fc region with an Fc receptor or ligand. Effector functions include FcγR-mediated effector functions such as ADCC and ADCP, and complement-mediated effector functions such as CDC. Further, effector functions include FcγRIlb-mediated effector functions, such as inhibitory functions (e.g., downregulating, reducing, inhibiting etc., B cell responses, e.g., a humoral immune response).

Fc or Fc region: The terms “Fc” or “Fc region,” or other grammatical equivalents as used herein is meant the polypeptide comprising the constant region of an antibody excluding the first constant region immunoglobulin domain and in some cases, part of the hinge. Thus, Fc may refer to the last two constant region immunoglobulin domains of IgG, and the flexible hinge N-terminal to these domains. For IgG, Fc comprises immunoglobulin domains Cgamma2 and Cgamma3 (Cγ2 and Cγ3) and the hinge between Cgamma1 (Cγ1) and Cgamma2. Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an Fc polypeptide, as described below.

Fc gamma receptor, or FcγR: The terms “Fc gamma receptor” or “FcγR” or other grammatical equivalents as used herein is meant any member of the family of proteins that bind the IgG antibody Fc region and are substantially encoded by the FcγR genes. In humans this family includes but is not limited to FcγRI (CD64), including isoforms FcγRIa, FcγRIb, and FcγRIc; FcγRII (CD32), including isoforms FcγRIla (including allotypes H131 and R131), FcγRIIb (including FcγRllb-1 and FcγRllb-2), and FcγRIIc; and FcγRI 11 (CD16), including isoforms FcγRI lla (including allotypes V1 58 and F158) and FcγRIIIb (including allotypes FcγRlllb-NA1 and FcγRl llb-NA2) (Jefferis et al., 2002, Immunol Lett 82:57-65, incorporated entirely by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγR 111 (CD16), and FcγRIII-2 (CD16-2), as well as any undiscovered mouse FcγRs or FcγR isoforms or allotypes.

Improve, increase, inhibit or reduce: As used herein, the terms “improve,” “increase” “inhibit” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein, e.g., a subject who is administered a placebo. A “control subject” is a subject afflicted with the same form of disease as the subject being treated, who is about the same age as the subject being treated.

Modification: The term “modification” or other grammatical equivalents herein is meant an alteration in the physical, chemical, or sequence properties of a protein, polypeptide, antibody, or immunoglobulin. Modifications described herein include amino acid modifications (including amino acid substitutions) and glycoform modifications.

Target Antigen: The term “target antigen” or other grammatical equivalents as used herein is meant the molecule that is bound by the variable region of a given antibody, or the fusion partner of an Fc fusion. A target antigen may be a protein, carbohydrate, lipid, or other chemical compound. An antibody or Fc fusion is said to be “specific” for a given target antigen based on having affinity for the target antigen. In some embodiments, the target antigen for obexelimab is CD19.

Treating: The term “treat,” “treatment,” or “treating” or other grammatical equivalents refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease. In some embodiments, the term “treating” or its grammatically equivalents refer to preventing a disease condition, an onset of one or more symptoms associated with the condition, or a significant increase or a significant decrease in the level of one or more biomarkers associated with the condition from its normal level.

Obexelimab: The term “Obexelimab” as used herein, is an Fc engineered humanized monoclonal antibody (mAb) that binds to the human B-cell restricted surface antigen CD19 and has enhanced Fc binding to Fcγ receptor IIb (FcγRIIb). The molecule is an IgG1 immunoglobulin with a kappa light chain and 2 amino acid substitutions in the constant portion of the heavy chain. Obexelimab is a monoclonal antibody with a projected mass of approximately 147,426 Da based on the amino acid sequence. The heavy and light chains of obexelimab are given by SEQ ID NO:10, and SEQ ID NO:9, respectively.

Relapse: “Relapse” or other grammatical equivalents refer to appearance of new or worsening symptoms, or the return of old symptoms associated with a disease or disorder. Those of skill in the art will appreciate that the terms attack, flare, or exacerbation may also be used to refer to the same concept.

Relapsing Multiple Sclerosis (RMS): “Relapsing multiple sclerosis” or “relapsing forms of MS”, or other grammatic equivalents, is a type of multiple sclerosis (MS) characterized by episodes of relapsing neurological symptoms (e.g., relapses), followed by periods of partial or complete recovery (e.g., remission). RMS typically includes two main subtypes, RRMS and active SPMS, and in some cases further includes CIS.

Therapeutically effective dosing regimen: As used herein, the term “therapeutically effective dosing regimen” or other grammatical equivalents of a therapeutic agent means an amount and frequency that are sufficient, when administered to a subject suffering from or susceptible to a disease, disorder, and/or condition, to treat, diagnose, prevent, and/or delay the onset of the symptom(s) of the disease, disorder, and/or condition. In some embodiments, the “therapeutically effective dosing regimen” is sufficient to prevent progression of a disease condition, an onset of one or more symptoms or complications associated with the condition, or a significant increase or a significant decrease in the level of one or more biomarkers associated with the condition from its normal level.

Any numerical values used in this application are meant to cover any variations within the standard deviation or normal fluctuations appreciated by one of ordinary skill in the relevant art.

### Multiple Sclerosis (MS)

MS is a chronic and potentially disabling immune mediated disease of the CNS characterized by the progressive destruction of the myelin. MS manifests diverse neurological symptoms such as motor paralysis, sensory impairment, higher brain dysfunction, visual loss, and dysuria due to infiltration of autoreactive lymphocytes (e.g., T cells or B cells) into the brain, the spinal cord, or the optic nerve, causing inflammations targeting perineural myelin proteins. MS patients develop transient and repetitive inflammations at various sites of the CNS. As each inflammation occurs, neurological symptoms are manifested depending on the inflammation site.

While long recognized as primarily a T-cell mediated disease, B cells also play an active role in the pathogenesis of MS although the disease mechanisms are not fully understood. B cell subsets play several roles in immunity, most of which are beneficial. For example, the B cell lineage is best known for making antibodies, a function not performed by B cells themselves but instead by activated cells that have differentiated into Plasma Cells. High affinity antibodies are central to protective, pathogen-specific immune responses to infection or following vaccination. However, while high-affinity auto-antigen (Ag)-specific antibodies do contribute to pathology in the MS-related disorders NMOSD and MOGSDs (Jain, R. W., et al., 2022. Nat. Rev. Immunol. 22:513-524), antibody production is not the primary B cell pathological contribution to MS. Indeed, while therapeutic anti-CD20 antibodies effectively eliminate B cells, they do not eliminate antibody-producing Plasma Cells, nor are antibody levels reduced within the therapeutic timeframe following administration of anti-CD20-based drugs (Graft et al., 2021, Lee et al., 2021, and Margoni et al., 2022). Therefore, B cells must have other functions that contribute to the ongoing inflammatory response that underlies disease progression.

One of the primary candidate mechanisms through which B cells may drive CNS autoimmunity is through the presentation of auto-Ag to T cells. This, combined with any cytokines that the B cell may produce at the same time, could directly activate naïve autoimmune T cells in lymphatic tissue and influence their differentiation into effector subsets. The process of Ag presentation requires the Ag presenting cell (APC) to collect and internalize protein Ags prior to processing them for loading on to major histocompatibility complex (MHC). This peptide:MHC combination is then transported to the cell surface and exposed to migrating T cells that sample local APCs for cognate Ag via their specific T cell Receptor (TCR).

While B cells are described in textbooks as one of the three “professional” APCs (along with Dendritic Cells and Macrophages), they are not well known as activators of T cells. In fact, their APC function is best understood in the context of the high affinity antibody response where B cell activation rather than that of T cells is the primary outcome. These events occur in specialized structures called Germinal Centers (GC) within secondary lymphoid tissues. In this case, naïve B cells that encounter an Ag that binds to their specific B cell receptor (BCR) internalize it (and anything physically linked to it) and presents it to already activated T follicular helper (Tfh) cells that are specific for the same antigen. These cognate interactions are necessary to fully activate the B cell and are foundational to the mechanisms that select high affinity B cells in the Germinal Center and for differentiation into Plasma Cells to produce antibody (Haberman, A. M., et al., 2019. Immunol. Rev. 288:10-27; Kerfoot, S. M., et al., 2011. Immunity 34:947-960; Jain, R. W., et al., 2018., Cell Rep. 25:3342-3355.e5; Parham, K. A., et al., 2022. J. Immunol. 209:1703-1712).

Other scenarios where B cells may present Ag to T cells resulting in T cell activation are much less well understood. Because B cells selectively acquire Ag via BCR binding, they are known to be able to concentrate low-concentration Ag much more effectively than other APCs, and for this reason have been implicated in the initiation of some autoimmune T cell responses (Rodríguez-Pinto, D. 2005. Cell. Immunol. 238:67-75). However, B cells that are specific for any given antigen are extremely rare, and typically B cells are physically separated from naïve T cells in lymphoid tissues, so it is not clear when or where within the lymphoid microanatomy such naïve T cell: cognate B cell interactions could occur.

While activated T cells readily infiltrate MS demyelinating lesions, B cells are relatively rare within the CNS parenchyma (Jain and Yong, 2022). Instead, considerable numbers of B cells can collect along with T cells in the meninges, often immediately adjacent to a lesion (Reali, C., R. et al., 2020. Brain Pathol. 30:779-793, Bell, L., et al., 2020. Front. Immunol. 10; Choi, S. R., et al., 2012. Brain J. Neurol. 135:2925-2937). These clusters of lymphocytes can sometimes become sufficiently organized to resemble lymphoid tissues with separate T cell zones and B cell follicles, but more often than not they are a disorganized mix of these cells. Because of their association with lesions and because their presence is associated with more severe disease (Jain and Yong, 2022), there has been a great deal of interest in these clusters as potential locations from which B cells could exert pathological function(s). Because they can resemble secondary lymphoid tissues, these structures may sustain the autoimmune anti-myelin response from behind the Blood Brain/Meningeal Barriers (Pipi, E. et al., 2018. Front. Immunol. 9). In this scenario, B cells may act as APCs to present locally-acquired CNS auto-Ag to recently recruited effector T cells, resulting in their reactivation to drive inflammation targeting oligodendrocytes in the local parenchyma. Therefore, B cells may function as APCs to drive the autoimmune responses from multiple locations, including the peripheral lymphatics and the meninges.

### Forms of Multiple Sclerosis

MS diagnosis involves a combination of clinical history, neurological exams, MRI findings, and sometimes cerebrospinal fluid analysis. Understanding the form of MS is essential for tailoring treatment and predicting disease progression. These diagnostic criteria rely upon the general principles of scattered lesions in the central white matter occurring at different times and not explained by other etiologies such as infection, vascular disorder, or autoimmune disorder (McDonald et al., Ann. Neurol. 50:121-7 (2001)).

To fulfill a diagnosis of MS based on the 2017 McDonald criteria (Thompson A J, Banwell B L, Barkhof F, et al. Diagnosis of multiple sclerosis: 2017 revisions of the McDonald criteria, Lancet Neurol. 2018;17 (2): 162-173.), an individual must have evidence of CNS damage that is disseminating in space, or appearing in multiple regions of the nervous system and evidence of damage that is disseminating in time or occurring at different points in time.

MS has several forms of disease: RRMS (80%- 85% of cases at onset), PPMS (10%- 15% at onset), progressive relapsing MS (PRMS; 5% at onset); and secondary progressive MS (SPMS) (Kremenchutzky et al. Brain 122 (Pt 10):1941-50 (1999); Confavreux et al. N Engl J Med 343(20): 1430-8 (2000)). Additionally, CIS is the first attack of MS symptoms, which may progress into more incidence and a full MS diagnosis.

### Clinically Isolated Syndrome

CIS occurs when the immune system attacks the brain, spinal cord or optic nerves and refers to a first episode of neurologic symptoms like those in multiple sclerosis. However, CIS may or may not go on to develop into MS. 60%- 80% of patients with brain lesions observed on MRI similar to those in MS display a second neurologic event and diagnosis of MS within several years. If CIS is not accompanied by MRI-detected brain lesions, the chance to develop MS within several years is 20%. In some embodiments, the present invention provides a method of a treating a patient with CIS.

### Relapsing Remitting Multiple Sclerosis

RRMS is the continued periodic inflammatory attacks on myelin, as well as the nerve fibers themselves that characterize MS. During these inflammatory attacks, activated immune cells cause localized areas of damage resulting in the symptoms of MS. Because the location of the damage is variable, no two patients have exactly the same symptoms. It is estimated that 50% of patients with RRMS will develop SPMS in 10 years (Weinshenker et al. Brain 112(Pt 1):133-46 (1989)). In some embodiments, the present invention provides a method of a treating a patient with RRMS.

### Secondary Progressive Multiple Sclerosis

SPMS is a secondary progressive course from RRMS, characterized by worsening neurologic function over time and increased disability. SPMS can be described as active, meaning with relapses and/or evidence of new MRI activity during a specified period of time, or inactive. SPMS can also present signs of progression, more disability over time with or without relapses or new MRI activity, or without progression. In some embodiments, the present invention provides a method of a treating a patient with SPMS.

### Primary Progressive Multiple Sclerosis

PPMS is a form of multiple sclerosis characterized by a steady progression of neurological symptoms and disability from the onset of the disease, without distinct relapses or remissions. PPMS takes longer to diagnose, requiring a minimum of 12 months of symptom progression, has fewer treatment options compared to MS, and has an average age of onset being 10 years older than RMS. Additionally, patients tend to experience more quality of life problems and require more assistance with everyday activities compared to other forms of MS. In some embodiments, the present invention provides a method of a treating a patient with PPMS.

### Progressive Relapsing Multiple Sclerosis

PRMS, patients experience both relapses and progressive disease from onset. PRMS is diagnosed when a patient with at least a year of progressive symptoms experiences one or more relapses. PRMS is the least common form of MS, affecting about 5% of people. In some embodiments, the present invention provides a method of a treating a patient with PRMS.

### Patients

In some embodiments, an anti-CD19 antibody (e.g., obexelimab, or a variant thereof) is used to treat a patient suffering from MS. In some embodiments, an anti-CD19 antibody (e.g., obexelimab, or a variant thereof) is used to treat a patient suffering from RMS. In some embodiments, an anti-CD19 antibody (e.g., obexelimab, or a variant thereof) is used to treat a patient suffering from RRMS. In some embodiments, an anti-CD19 antibody (e.g., obexelimab, or a variant thereof) is used to treat a patient suffering from SPMS. In some embodiments, an anti-CD19 antibody (e.g., obexelimab, or a variant thereof) is used to treat a patient suffering from PPMS. In some embodiments, an anti-CD19 antibody (e.g., obexelimab, or a variant thereof) is used to treat a patient suffering from PRMS. In some embodiments, an anti-CD19 antibody (e.g., obexelimab, or a variant thereof) is used to treat a patient suffering from CIS.

In some embodiments, an anti-CD19 antibody (e.g., obexelimab, or a variant thereof) is used to treat a patient presenting an EDSS of ≤5.5. In some embodiments, a patient has an EDSS score of ≤5.0 prior to commencing treatment. In some embodiments, a patient has an EDSS score of ≤4.5 prior to commencing treatment. In some embodiments, a patient has an EDSS score of ≤4.0 prior to commencing treatment. In some embodiments, a patient has an EDSS score of ≤3.5 prior to commencing treatment. In some embodiments, a patient has an EDSS score of ≤3.0 prior to commencing treatment. In some embodiments, a patient has an EDSS score of ≤2.5 prior to commencing treatment. In some embodiments, a patient has an EDSS score of ≤2.0 prior to commencing treatment. In some embodiments, a patient has an EDSS score of ≤1.5 prior to commencing treatment.

In some embodiments, an anti-CD19 antibody (e.g., obexelimab, or a variant thereof) is used to treat a patient presenting an EDSS of ≥5.0. In some embodiments, a patient has an EDSS score of ≥5.0 prior to commencing treatment. In some embodiments, a patient has an EDSS score of ≥5.5 prior to commencing treatment. In some embodiments, a patient has an EDSS score of ≥6.0 prior to commencing treatment. In some embodiments, a patient has an EDSS score of ≥6.5 prior to commencing treatment. In some embodiments, a patient has an EDSS score of ≥7.0 prior to commencing treatment. In some embodiments, a patient has an EDSS score of ≥7.5 prior to commencing treatment. In some embodiments, a patient has an EDSS score of ≥8.0 prior to commencing treatment. In some embodiments, a patient has an EDSS score of ≥8.5 prior to commencing treatment. In some embodiments, a patient has an EDSS score of ≥9.0 prior to commencing treatment. In some embodiments, a patient has an EDSS score of ≥9.5 prior to commencing treatment.

In some embodiments, an anti-CD19 antibody (e.g., obexelimab, or a variant thereof) is used to treat a patient presenting at least 1 relapse within the previous year. In some embodiments, an anti-CD19 antibody (e.g., obexelimab, or a variant thereof) is used to treat a patient presenting ≥2 relapses within the past 2 years. In some embodiments, an anti-CD19 antibody (e.g., obexelimab, or a variant thereof) is used to treat a patient presenting ≥1 active Gd-enhancing brain lesion on an MRI scan within the past 6 months prior to screening. In some embodiments, an anti-CD19 antibody (e.g., obexelimab, or a variant thereof) is used to treat a patient presenting ≥1 active Gd-enhancing brain lesion on an MRI scan within the past year prior to screening.

### Anti-CD19 Antibodies (e.g., Obexelimab)

In some embodiments, an anti-CD19 antibody (e.g., obexelimab, or a variant thereof) is used to treat a patient suffering from RMS. In one aspect, the present invention provides a method of administering obexelimab for the treatment of RMS.

Obexelimab (XmAb5871) is a humanized anti-CD19 mAb with an Fc portion engineered for increased affinity to FcγRIIb, the only Fc receptor on B-cells. Obexelimab is a mAb specific for CD19 comprising: a light chain comprising a variable region having:
a CDR1 comprising RSSKSLQNVNGNTYLY (SEQ ID NO:2),
a CDR2 comprising RMSNLNS (SEQ ID NO:3), and
a CDR3 comprising MQHLEYPIT (SEQ ID NO:4); and
a heavy chain comprising a variable region having
a CDR1 comprising SYVMH (SEQ ID NO:5),
a CDR2 comprising WIGYINPYNDGTKY (SEQ ID NO:6), and
a CDR3 comprising GTYYYGTRVFDY (SEQ ID NO:7),
wherein the heavy chain comprises amino acid substitutions in the Fc region S267E and L328F as compared to SEQ ID NO:8:
wherein the numbering is according to the EU index, as in Kabat.

(SEQ ID NO: 8)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

Obexelimab works by exploiting the regulation of B-cell receptor (BCR) signaling by FcγRIIb. Obexelimab binds CD19 of the BCR complex and its Fc is engineered to increase its affinity for the inhibitory FcγRIIb. Since CD19 is associated with the BCR, Obexelimab tethering of CD19 to FcγRIIb on the same cell poises the BCR complex for inhibition upon antigen-induced BCR aggregation. Obexelimab capitalizes upon the natural inhibitory mechanism of FcγRIIb, the only Fc receptor expressed by B cells, which acts as a negative regulator in conditions of antigen excess and immune complex formation (Chu et al., 2014). Obexelimab may also have an improved safety profile compared to B cell depleting antibodies as it may not mediate B cell killing.

In some embodiments, obexelimab comprises: a light chain comprising amino acid sequence:

(SEQ ID NO: 9)
DIVMTQSPATLSLSPGERATLSCRSSKSLQNVNGNTYLYWFQQKPGQSP
QLLIYRMSNLNSGVPDRFSGSGSGTEFTLTISSLEPEDFAVYYCMQHLE
YPITFGAGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSENRGEC;

and heavy chain comprising an amino acid sequence:

(SEQ ID NO: 10)
EVQLVESGGGLVKPGGSLKLSCAASGYTFTSYVMHWVRQAPGKGLEWIG
YINPYNDGTKYNEKFQGRVTISSDKSISTAYMELSSLRSEDTAMYYCAR
GTYYYGTRVFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVEHEDPEVKENWYVDGVEVHNAKTK
PREEQYNSTYRVVSVLTVLHQDWLNGKEYKCKVSNKAFPAPIEKTISKA
KGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYT
QKSLSLSPGK

TABLE 1
Sequence of Heavy chain and Light chain amino
acid sequence of obexelimab

| Heavy Chain | Light Chain |
|---|---|
| EVQLVESGGGLVKPGGSLKLSCAASGYTFT | DIVMTQSPATLSLSPGERATLSCRSSKSLQNVN |
| SYVMHWVRQAPGKGLEWIGYINPYNDGTKY | GNTYLYWFQQKPGQSPQLLIYRMSNLNSGVPDR |
| NEKFQGRVTISSDKSISTAYMELSSLRSED | FSGSGSGTEFTLTISSLEPEDFAVYYCMQHLEY |
| TAMYYCARGTYYYGTRVFDYWGQGTLVTVS | PITFGAGTKLEIKRTVAAPSVFIFPPSDEQLKS |
| SASTKGPSVFPLAPSSKSTSGGTAALGCLV | GTASVVCLLNNFYPREAKVQWKVDNALQSGNSQ |
| KDYFPEPVTVSWNSGALTSGVHTFPAVLQS | ESVTEQDSKDSTYSLSSTLTLSKADYEKHKVYA |
| SGLYSLSSVVTVPSSSLGTQTYICNVNHKP | CEVTHQGLSSPVTKSENRGEC (SEQ ID |
| SNTKVDKKVEPKSCDKTHTCPPCPAPELLG | NO : 9) |
| GPSVFLFPPKPKDTLMISRTPEVTCVVVDV | |
| EHEDPEVKFNWYVDGVEVHNAKTKPREEQY | |
| NSTYRVVSVLTVLHQDWLNGKEYKCKVSNK | |
| AFPAPIEKTISKAKGQPREPQVYTLPPSRE | |
| EMTKNQVSLTCLVKGFYPSDIAVEWESNGQ | |
| PENNYKTTPPVLDSDGSFFLYSKLTVDKSR | |
| WQQGNVFSCSVMHEALHNHYTQKSLSLSPG | |
| K (SEQ ID NO: 10) | |
| Heavy Chain Variable Region (VH) | Light Chain Variable Region (VL) |
| EVQLVESGGGLVKPGGSLKLSCAASGYTFT | DIVMTQSPATLSLSPGERATLSCRSSKSLQNVN |
| SYVMHWVRQAPGKGLEWIGYINPYNDGTKY | GNTYLYWFQQKPGQSPQLLIYRMSNLNSGVPDR |
| NEKFQGRVTISSDKSISTAYMELSSLRSED | FSGSGSGTEFTLTISSLEPEDFAVYYCMQHLEY |
| TAMYYCARGTYYYGTRVFDYWGQGTLVTVS | PITFGAGTKLEIK (SEQ ID NO: 11) |
| S (SEQ ID NO: 12) | |

TABLE 2
Kabat heavy chain and light
chain CDRs of obexelimab.

| CDR | Amino Acid Sequence |
|---|---|
| LCDR1 | RSSKSLQNVNGNTYLY (SEQ ID NO: 2) |
| LCDR2 | RMSNLNS (SEQ ID NO: 3) |
| LCDR3 | MQHLEYPIT (SEQ ID NO: 4) |
| HCDR1 | SYVMH (SEQ ID NO: 5) |
| HCDR2 | WIGYINPYNDGTKY (SEQ ID NO: 6) |
| HCDR3 | GTYYYGTRVEDY (SEQ ID NO: 7) |

In some embodiments, obexelimab comprises a light chain variable region and a heavy chain variable region as given by Table 1. In some embodiments, obexelimab comprises CDRs as given by Table 2.

In some embodiments, an anti-CD19 antibody comprises a light chain comprising an amino acid sequence of SEQ ID NO:9. In some embodiments, an anti-CD19 antibody comprises a heavy chain comprising an amino acid sequence of SEQ ID NO:10. In some embodiments, an anti-CD19 antibody comprises a light chain and a heavy chain, wherein the light chain comprises an amino acid sequence of SEQ ID NO:9, and wherein the heavy chain comprises an amino acid sequence of SEQ ID NO:10.

In some embodiments, an anti-CD19 antibody comprises a light chain, wherein the light chain comprises a light chain variable region comprising an amino acid sequence of SEQ ID NO:11. In some embodiments, an anti-CD19 antibody comprises a heavy chain, wherein the heavy chain comprises a heavy chain variable region comprising an amino acid sequence of SEQ ID NO:12. In some embodiments, an anti-CD19 antibody comprises a light chain and a heavy chain, wherein the light chain comprises a light chain variable region comprising an amino acid sequence of SEQ ID NO:11, and wherein the heavy chain comprises a heavy chain variable region comprising an amino acid sequence of SEQ ID NO:12. In some embodiments, an anti-CD19 antibody comprises a heavy chain, wherein the heavy chain comprises a heavy chain variable region comprising an amino acid sequence of SEQ ID NO:12, and wherein the heavy chain further comprises an Fc region comprising amino acid substitutions S267E and L328F. In some embodiments, an anti-CD19 antibody comprises a light chain and a heavy chain, wherein the light chain comprises a light chain variable region comprising an amino acid sequence of SEQ ID NO:11, and wherein the heavy chain comprises a heavy chain variable region comprising an amino acid sequence of SEQ ID NO:12, and wherein the heavy chain further comprises an Fc region comprising amino acid substitutions S267E and L328F.

In some embodiments, an anti-CD19 antibody comprises a light chain comprising a LCDR1 comprising an amino acid sequence of SEQ ID NO:2, a LCDR2 comprising an amino acid sequence of SEQ ID NO:3, and a LCDR3 comprising an amino acid sequence of SEQ ID NO:4. In some embodiments, an anti-CD19 antibody comprises a heavy chain comprising a HCDR1 comprising an amino acid sequence of SEQ ID NO:5, a HCDR2 comprising an amino acid sequence of SEQ ID NO:6, and a HCDR3 comprising an amino acid sequence of SEQ ID NO:7. In some embodiments, an anti-CD19 antibody comprises a heavy chain comprising a HCDR1 comprising an amino acid sequence of SEQ ID NO:5, a HCDR2 comprising an amino acid sequence of SEQ ID NO:6, and a HCDR3 comprising an amino acid sequence of SEQ ID NO:7, and wherein the heavy chain further comprises an Fc region comprising amino acid substitutions S267E and L328F. In some embodiments, an anti-CD19 antibody comprises a light chain and heavy chain, wherein the light chain comprises a LCDR1 comprising an amino acid sequence of SEQ ID NO:2, a LCDR2 comprising an amino acid sequence of SEQ ID NO:3, and a LCDR3 comprising an amino acid sequence of SEQ ID NO:4, and wherein the heavy chain comprises a HCDR1 comprising an amino acid sequence of SEQ ID NO:5, a HCDR2 comprising an amino acid sequence of SEQ ID NO:6, and a HCDR3 comprising an amino acid sequence of SEQ ID NO:7. In some embodiments, an anti-CD19 antibody comprises a light chain and heavy chain, wherein the light chain comprises a LCDR1 comprising an amino acid sequence of SEQ ID NO:2, a LCDR2 comprising an amino acid sequence of SEQ ID NO:3, and a LCDR3 comprising an amino acid sequence of SEQ ID NO:4, and wherein the heavy chain comprises a HCDR1 comprising an amino acid sequence of SEQ ID NO:5, a HCDR2 comprising an amino acid sequence of SEQ ID NO:6, and a HCDR3 comprising an amino acid sequence of SEQ ID NO:7, and wherein the heavy chain further comprises an Fc region comprising amino acid substitutions S267E and L328F.

### Variants

In some embodiments, a variant of obexelimab is an immunoglobulin specific for CD19 comprises: a light chain comprising a variable region having a CDR1 comprising RSSKSLQNVNGNTYLY, a CDR2 comprising RMSNLNS, and a CDR3 comprising MQHLEYPIT; and a heavy chain comprising a variable region having a CDR1 comprising SYVMH, a CDR2 comprising WIGYINPYNDGTKY, and a CDR3 comprising GTYYYGTRVFDY, wherein the heavy chain comprises amino acid substitutions in the Fc region S267E and L328F as compared to:

(SEQ ID NO: 8)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

wherein the numbering is according to the EU index, as in Kabat.

In some embodiments, a variant of obexelimab comprises a heavy chain variable region (VH) and/or a light chain variable region (VL), comprising a CDR1, a CDR2, and a CDR3, each of which differs by no more than 1, 2, 3, 4 or 5 amino acid residues from each of RSSKSLQNVNGNTYLY (SEQ ID NO:2), RMSNLNS (SEQ ID NO:3), MQHLEYPIT (SEQ ID NO:4), SYVMH (SEQ ID NO:5), WIGYINPYNDGTKY (SEQ ID NO:6) and/or GTYYYGTRVFDY (SEQ ID NO:7). In some embodiments, a variant of obexelimab comprises a heavy chain, wherein the heavy chain comprises an Fc region comprising amino acid substitutions S267E and L328F.

In some embodiments, an anti-CD19 antibody comprises a light chain comprising a LCDR1, LCDR2, and LCDR3, each of which differs by no more than 1 residue from each of SEQ ID NO:2, SEQ ID NO:3, and SEQ ID NO:4. In some embodiments, an anti-CD19 antibody comprises a light chain comprising a LCDR1, LCDR2, and LCDR3, each of which differs by no more than 2 residues from each of SEQ ID NO:2, SEQ ID NO:3, and SEQ ID NO:4. In some embodiments, an anti-CD19 antibody comprises a light chain comprising a LCDR1, LCDR2, and LCDR3, each of which differs by no more than 3 residues from each of SEQ ID NO:2, SEQ ID NO:3, and SEQ ID NO:4. In some embodiments, an anti-CD19 antibody comprises a light chain comprising a LCDR1, LCDR2, and LCDR3, each of which differs by no more than 4 residue from each of SEQ ID NO:2, SEQ ID NO:3, and SEQ ID NO:4. In some embodiments, an anti-CD19 antibody comprises a light chain comprising a LCDR1, LCDR2, and LCDR3, each of which differs by no more than 5 residues from each of SEQ ID NO:2, SEQ ID NO:3, and SEQ ID NO:4.

In some embodiments, an anti-CD19 antibody comprises a heavy chain comprising a HCDR1, HCDR2, and HCDR3, each of which differs by no more than 1 residue from each of SEQ ID NO:5, SEQ ID NO:6, and SEQ ID NO:7. In some embodiments, an anti-CD19 antibody comprises a heavy chain comprising a HCDR1, HCDR2, and HCDR3, each of which differs by no more than 2 residues from each of SEQ ID NO:5, SEQ ID NO:6, and SEQ ID NO:7. In some embodiments, an anti-CD19 antibody comprises a heavy chain comprising a HCDR1, HCDR2, and HCDR3, each of which differs by no more than 3 residues from each of SEQ ID NO:5, SEQ ID NO:6, and SEQ ID NO:7. In some embodiments, an anti-CD19 antibody comprises a heavy chain comprising a HCDR1, HCDR2, and HCDR3, each of which differs by no more than 4 residues from each of SEQ ID NO:5, SEQ ID NO:6, and SEQ ID NO:7. In some embodiments, an anti-CD19 antibody comprises a heavy chain comprising a HCDR1, HCDR2, and HCDR3, each of which differs by no more than 5 residues from each of SEQ ID NO:5, SEQ ID NO:6, and SEQ ID NO:7.

In some embodiments, an anti-CD19 antibody comprises a heavy chain comprising a HCDR1, HCDR2, and HCDR3, each of which differs by no more than 1 residue from each of SEQ ID NO:5, SEQ ID NO:6, and SEQ ID NO:7, and wherein the heavy chain further comprises an Fc region comprising amino acid substitutions S267E and L328F. In some embodiments, an anti-CD19 antibody comprises a heavy chain comprising a HCDR1, HCDR2, and HCDR3, each of which differs by no more than 2 residues from each of SEQ ID NO:5, SEQ ID NO:6, and SEQ ID NO:7, and wherein the heavy chain further comprises an Fc region comprising amino acid substitutions S267E and L328F. In some embodiments, an anti-CD19 antibody comprises a heavy chain comprising a HCDR1, HCDR2, and HCDR3, each of which differs by no more than 3 residues from each of SEQ ID NO:5, SEQ ID NO:6, and SEQ ID NO:7, and wherein the heavy chain further comprises an Fc region comprising amino acid substitutions S267E and L328F. In some embodiments, an anti-CD19 antibody comprises a heavy chain comprising a HCDR1, HCDR2, and HCDR3, each of which differs by no more than 4 residues from each of SEQ ID NO:5, SEQ ID NO:6, and SEQ ID NO:7, and wherein the heavy chain further comprises an Fc region comprising amino acid substitutions S267E and L328F. In some embodiments, an anti-CD19 antibody comprises a heavy chain comprising a HCDR1, HCDR2, and HCDR3, each of which differs by no more than 5 residues from each of SEQ ID NO:5, SEQ ID NO:6, and SEQ ID NO:7, and wherein the heavy chain further comprises an Fc region comprising amino acid substitutions S267E and L328F.

In some embodiments, the variant of obexelimab comprises: a light chain variable region comprising an amino acid sequence that is at least 90%, 91%, 92%, 93%, 94%, 95%, 96%, 97%, 98% or 99% identical to the light chain variable region identified in Table 1. In some embodiments, the variant of obexelimab comprises: a heavy chain variable region comprising an amino acid sequence that is at least 90%, 91%, 92%, 93%, 94%, 95%, 96%, 97%, 98% or 99% identical to the heavy chain variable region as identified in Table 1.

In some embodiments, an anti-CD19 antibody comprises a light chain comprising a light chain variable region at least 80% identical to SEQ ID NO:11. In some embodiments, an anti-CD19 antibody comprises a light chain comprising a light chain variable region at least 85% identical to SEQ ID NO:11. In some embodiments, an anti-CD19 antibody comprises a light chain comprising a light chain variable region at least 90% identical to SEQ ID NO:11. In some embodiments, an anti-CD19 antibody comprises a light chain comprising a light chain variable region at least 91% identical to SEQ ID NO:11. In some embodiments, an anti-CD19 antibody comprises a light chain comprising a light chain variable region at least 92% identical to SEQ ID NO:11. In some embodiments, an anti-CD19 antibody comprises a light chain comprising a light chain variable region at least 93% identical to SEQ ID NO:11. In some embodiments, an anti-CD19 antibody comprises a light chain comprising a light chain variable region at least 94% identical to SEQ ID NO:11. In some embodiments, an anti-CD19 antibody comprises a light chain comprising a light chain variable region at least 95% identical to SEQ ID NO:11. In some embodiments, an anti-CD19 antibody comprises a light chain comprising a light chain variable region at least 96% identical to SEQ ID NO:11. In some embodiments, an anti-CD19 antibody comprises a light chain comprising a light chain variable region at least 97% identical to SEQ ID NO:11. In some embodiments, an anti-CD19 antibody comprises a light chain comprising a light chain variable region at least 98% identical to SEQ ID NO:11. In some embodiments, an anti-CD19 antibody comprises a light chain comprising a light chain variable region at least 99% identical to SEQ ID NO:11.

In some embodiments, an anti-CD19 antibody comprises a heavy chain comprising a heavy chain variable region at least 80% identical to SEQ ID NO:12. In some embodiments, an anti-CD19 antibody comprises a heavy chain comprising a heavy chain variable region at least 85% identical to SEQ ID NO:12. In some embodiments, an anti-CD19 antibody comprises a heavy chain comprising a heavy chain variable region at least 90% identical to SEQ ID NO:12. In some embodiments, an anti-CD19 antibody comprises a heavy chain comprising a heavy chain variable region at least 91% identical to SEQ ID NO:12. In some embodiments, an anti-CD19 antibody comprises a heavy chain comprising a heavy chain variable region at least 92% identical to SEQ ID NO:12. In some embodiments, an anti-CD19 antibody comprises a heavy chain comprising a heavy chain variable region at least 93% identical to SEQ ID NO:12. In some embodiments, an anti-CD19 antibody comprises a heavy chain comprising a heavy chain variable region at least 94% identical to SEQ ID NO:12. In some embodiments, an anti-CD19 antibody comprises a heavy chain comprising a heavy chain variable region at least 95% identical to SEQ ID NO:12. In some embodiments, an anti-CD19 antibody comprises a heavy chain comprising a heavy chain variable region at least 96% identical to SEQ ID NO:12. In some embodiments, an anti-CD19 antibody comprises a heavy chain comprising a heavy chain variable region at least 97% identical to SEQ ID NO:12. In some embodiments, an anti-CD19 antibody comprises a heavy chain comprising a heavy chain variable region at least 98% identical to SEQ ID NO:12. In some embodiments, an anti-CD19 antibody comprises a heavy chain comprising a heavy chain variable region at least 99% identical to SEQ ID NO:12.

In some embodiments, the variant of obexelimab comprises: a light chain variable region comprising an amino acid sequence that is at least 90%, 91%, 92%, 93%, 94%, 95%, 96%, 97%, 98% or 99% identical to the light chain variable region identified in Table 1 and a heavy chain variable region comprising an amino acid sequence that is at least 90%, 91%, 92%, 93%, 94%, 95%, 96%, 97%, 98% or 99% identical to the heavy chain variable region as identified in Table 1 and further comprises amino acid substitutions in the Fc region S267E and L328F as compared to SEQ ID NO:8, wherein the numbering is according to the EU index.

In some embodiments, an anti-CD19 antibody comprises a light chain and a heavy chain, wherein the light chain comprises a light chain variable region at least 80% identical to SEQ ID NO:11, and wherein the heavy chain comprises a heavy chain variable region at least 80% identical to SEQ ID NO:12. In some embodiments, an anti-CD19 antibody comprises a light chain and a heavy chain, wherein the light chain comprises a light chain variable region at least 85% identical to SEQ ID NO:11, and wherein the heavy chain comprises a heavy chain variable region at least 85% identical to SEQ ID NO:12. In some embodiments, an anti-CD19 antibody comprises a light chain and a heavy chain, wherein the light chain comprises a light chain variable region at least 90% identical to SEQ ID NO:11, and wherein the heavy chain comprises a heavy chain variable region at least 90% identical to SEQ ID NO:12. In some embodiments, an anti-CD19 antibody comprises a light chain and a heavy chain, wherein the light chain comprises a light chain variable region at least 91% identical to SEQ ID NO:11, and wherein the heavy chain comprises a heavy chain variable region at least 91% identical to SEQ ID NO:12. In some embodiments, an anti-CD19 antibody comprises a light chain and a heavy chain, wherein the light chain comprises a light chain variable region at least 92% identical to SEQ ID NO:11, and wherein the heavy chain comprises a heavy chain variable region at least 92% identical to SEQ ID NO:12. In some embodiments, an anti-CD19 antibody comprises a light chain and a heavy chain, wherein the light chain comprises a light chain variable region at least 93% identical to SEQ ID NO:11, and wherein the heavy chain comprises a heavy chain variable region at least 93% identical to SEQ ID NO:12. In some embodiments, an anti-CD19 antibody comprises a light chain and a heavy chain, wherein the light chain comprises a light chain variable region at least 94% identical to SEQ ID NO:11, and wherein the heavy chain comprises a heavy chain variable region at least 94% identical to SEQ ID NO:12. In some embodiments, an anti-CD19 antibody comprises a light chain and a heavy chain, wherein the light chain comprises a light chain variable region at least 95% identical to SEQ ID NO:11, and wherein the heavy chain comprises a heavy chain variable region at least 95% identical to SEQ ID NO:12. In some embodiments, an anti-CD19 antibody comprises a light chain and a heavy chain, wherein the light chain comprises a light chain variable region at least 96% identical to SEQ ID NO:11, and wherein the heavy chain comprises a heavy chain variable region at least 96% identical to SEQ ID NO:12. In some embodiments, an anti-CD19 antibody comprises a light chain and a heavy chain, wherein the light chain comprises a light chain variable region at least 97% identical to SEQ ID NO:11, and wherein the heavy chain comprises a heavy chain variable region at least 97% identical to SEQ ID NO:12. In some embodiments, an anti-CD19 antibody comprises a light chain and a heavy chain, wherein the light chain comprises a light chain variable region at least 98% identical to SEQ ID NO:11, and wherein the heavy chain comprises a heavy chain variable region at least 98% identical to SEQ ID NO:12. In some embodiments, an anti-CD19 antibody comprises a light chain and a heavy chain, wherein the light chain comprises a light chain variable region at least 99% identical to SEQ ID NO:11, and wherein the heavy chain comprises a heavy chain variable region at least 99% identical to SEQ ID NO:12.

In some embodiments, the variant of obexelimab comprises: a light chain comprising an amino acid sequence 70%, 75%, 80%, 85%, 90%, 95%, 98% or 99% identical to the light chain as identified in Table 1. In some embodiments, the variant of obexelimab comprises: a heavy chain comprising an amino acid sequence 70%, 75%, 80%, 85%, 90%, 95%, 98% or 99% identical to the heavy chain as identified in Table 1.

In some embodiments, an anti-CD19 antibody comprises a light chain amino acid sequence that is at least 70% identical to SEQ ID NO:9. In some embodiments, an anti-CD19 antibody comprises a light chain amino acid sequence that is at least 75% identical to SEQ ID NO:9. In some embodiments, an anti-CD19 antibody comprises a light chain amino acid sequence that is at least 80% identical to SEQ ID NO:9. In some embodiments, an anti-CD19 antibody comprises a light chain amino acid sequence that is at least 85% identical to SEQ ID NO:9. In some embodiments, an anti-CD19 antibody comprises a light chain amino acid sequence that is at least 90% identical to SEQ ID NO:9. In some embodiments, an anti-CD19 antibody comprises a light chain amino acid sequence that is at least 91% identical to SEQ ID NO:9. In some embodiments, an anti-CD19 antibody comprises a light chain amino acid sequence that is at least 92% identical to SEQ ID NO:9. In some embodiments, an anti-CD19 antibody comprises a light chain amino acid sequence that is at least 93% identical to SEQ ID NO:9. In some embodiments, an anti-CD19 antibody comprises a light chain amino acid sequence that is at least 94% identical to SEQ ID NO:9. In some embodiments, an anti-CD19 antibody comprises a light chain amino acid sequence that is at least 95% identical to SEQ ID NO:9. In some embodiments, an anti-CD19 antibody comprises a light chain amino acid sequence that is at least 96% identical to SEQ ID NO:9. In some embodiments, an anti-CD19 antibody comprises a light chain amino acid sequence that is at least 97% identical to SEQ ID NO:9. In some embodiments, an anti-CD19 antibody comprises a light chain amino acid sequence that is at least 98% identical to SEQ ID NO:9. In some embodiments, an anti-CD19 antibody comprises a light chain amino acid sequence that is at least 99% identical to SEQ ID NO:9.

In some embodiments, an anti-CD19 antibody comprises a heavy chain amino acid sequence that is at least 70% identical to SEQ ID NO:10. In some embodiments, an anti-CD19 antibody comprises a heavy chain amino acid sequence that is at least 75% identical to SEQ ID NO:10. In some embodiments, an anti-CD19 antibody comprises a heavy chain amino acid sequence that is at least 80% identical to SEQ ID NO:10. In some embodiments, an anti-CD19 antibody comprises a heavy chain amino acid sequence that is at least 85% identical to SEQ ID NO:10. In some embodiments, an anti-CD19 antibody comprises a heavy chain amino acid sequence that is at least 90% identical to SEQ ID NO:10. In some embodiments, an anti-CD19 antibody comprises a heavy chain amino acid sequence that is at least 91% identical to SEQ ID NO:10. In some embodiments, an anti-CD19 antibody comprises a heavy chain amino acid sequence that is at least 92% identical to SEQ ID NO:10. In some embodiments, an anti-CD19 antibody comprises a heavy chain amino acid sequence that is at least 93% identical to SEQ ID NO:10. In some embodiments, an anti-CD19 antibody comprises a heavy chain amino acid sequence that is at least 94% identical to SEQ ID NO:10. In some embodiments, an anti-CD19 antibody comprises a heavy chain amino acid sequence that is at least 95% identical to SEQ ID NO:10. In some embodiments, an anti-CD19 antibody comprises a heavy chain amino acid sequence that is at least 96% identical to SEQ ID NO:10. In some embodiments, an anti-CD19 antibody comprises a heavy chain amino acid sequence that is at least 97% identical to SEQ ID NO:10. In some embodiments, an anti-CD19 antibody comprises a heavy chain amino acid sequence that is at least 98% identical to SEQ ID NO:10. In some embodiments, an anti-CD19 antibody comprises a heavy chain amino acid sequence that is at least 99% identical to SEQ ID NO:10.

In some embodiments, the variant of obexelimab comprises: a light chain comprising an amino acid sequence that is at least 90%, 91%, 92%, 93%, 94%, 95%, 96%, 97%, 98% or 99% identical to the light chain sequence as identified in Table 1 and a heavy chain comprising an amino acid sequence that is at least 90%, 91%, 92%, 93%, 94%, 95%, 96%, 97%, 98% or 99% identical to the heavy chain sequence as identified in Table 1, and wherein the heavy chain of the variant further comprises amino acid substitutions in the Fc region S267E and L328F as compared to SEQ ID NO:8, wherein the numbering is according to the EU index, as in Kabat.

In some embodiments, a suitable variant of obexelimab binds to the same epitope on human CD19, as an antibody comprising a light chain and a heavy chain as identified in Table 1. Epitope binding may be determined by a method known in the art.

In some embodiments, a suitable variant of obexelimab competes for binding to human CD19, as an antibody comprising a light chain and heavy chain as identified in Table 1, under a binning assay known in the art. As used herein, a binning assay refers to any method to regionally map the epitope to which the antibody binds. Standard methods for such antibody characterization, also known as epitope binning, typically involve surface plasmon resonance (SPR) technology. Using SPR, mAb candidates are screened pairwise for binding to a target protein. Other standard methods involve ELISA-based screens and may require synthesis of sets of overlapping peptides corresponding to the protein of interest.

In some embodiments, the human CD19 comprises an amino acid sequence of SEQ ID NO:1. In some embodiments, an anti-CD19 antibody binds to the extracellular domain of human CD19.

(SEQ ID NO: 1)
MPPPRLLFFLLFLTPMEVRPEEPLVVKVEEGDNAVLQCLKGTSDGPTQQ
LTWSRESPLKPFLKLSLGLPGLGIHMRPLAIWLFIFNVSQQMGGFYLCQ
PGPPSEKAWQPGWTVNVEGSGELFRWNVSDLGGLGCGLKNRSSEGPSSP
SGKLMSPKLYVWAKDRPEIWEGEPPCLPPRDSLNQSLSQDLTMAPGSTL
WLSCGVPPDSVSRGPLSWTHVHPKGPKSLLSLELKDDRPARDMWVMETG
LLLPRATAQDAGKYYCHRGNLTMSFHLEITARPVLWHWLLRTGGWKVSA
VTLAYLIFCLCSLVGILHLQRALVLRRKRKRMTDPTRRFFKVTPPPGSG
PQNQYGNVLSLPTPTSGLGRAQRWAAGLGGTAPSYGNPSSDVQADGALG
SRSPPGVGPEEEEGEGYEEPDSEEDSEFYENDSNLGQDQLSQDGSGYEN
PEDEPLGPEDEDSFSNAESYENEDEELTQPVARTMDFLSPHGSAWDPSR
EATSLGSQSYEDMRGILYAAPQLRSIRGQPGPNHEEDADSYENMDNPDG
PDPAWGGGGRMGTWSTR

### Fc Receptor Binding Properties

Anti-CD19 antibodies (e.g., obexelimab) disclosed herein comprise an Fc variant that has enhanced Fc binding to the inhibitory Fcγ receptor IIb (FcγRIIb). FcγRIIb, the only FcR on B cells, serves as an antibody-sensing down-regulator of humoral immunity that is naturally engaged by immune complexes. When sufficient antibody is raised against a given antigen, specific immune complexes form and co-engage FcγRIIb and BCR with high avidity, selectively suppressing only B cells recognizing cognate antigen. In addition, FcγRIIb regulates the activity of other B cell stimulators including interleukin (IL)-4, LPS, and BAFF that amplify BCR-driven proliferation and differentiation. By simultaneously binding CD19 and FcγRIIb, obexelimab (and variants described herein) mimics the action of antigen-antibody complexes and down-regulates B cell activity.

The Fc variants disclosed herein may be optimized for a variety of Fc receptor binding properties. An Fc variant that is engineered or predicted to display one or more optimized properties is herein referred to as an “optimized Fc variant.” Properties that may be optimized include but are not limited to enhanced or reduced affinity for an FcγR. In one embodiment, the Fc variants disclosed herein are optimized to possess enhanced affinity for an inhibitory receptor FcγRIIb. In other embodiments, immunoglobulins disclosed herein provide enhanced affinity for FcγRIIb, yet reduced affinity for one or more activating FcγRs, including for example FcγRI, FcγRIIa, FcγRIIIa, and/or FcγRIIIb. The FcγR receptors may be expressed on cells from any organism, including but not limited to human, cynomolgus monkeys, and mice. The Fc variants disclosed herein may be optimized to possess enhanced affinity for human FcγRIIb.

An Fc variant comprises one or more amino acid modifications relative to a parent Fc polypeptide, wherein the amino acid modification(s) provide one or more optimized properties. An Fc variant disclosed herein differs in amino acid sequence from its parent by virtue of at least one amino acid modification. Thus, Fc variants disclosed herein have at least one amino acid modification compared to the parent. Alternatively, the Fc variants disclosed herein may have more than one amino acid modification as compared to the parent, for example from two to fifty amino acid modifications, e.g., from two to ten amino acid modifications, from two to five amino acid modifications, etc. compared to the parent. Thus, the sequences of the Fc variants and those of the parent Fc polypeptide are substantially homologous. For example, the variant Fc variant sequences herein will possess at least 80% homology with the parent Fc variant sequence, e.g., at least 90% homology, at least 95% homology, at least 98% homology, at least 99% homology, etc. Modifications disclosed herein include amino acid modifications, including insertions, deletions, and substitutions. Modifications disclosed herein also include glycoform modifications.

Modifications may be made genetically using molecular biology or may be made enzymatically or chemically.

Fc variants disclosed herein are defined according to the amino acid modifications that compose them. Thus, for example, S267E is an Fc variant with the substitution S267E relative to the parent Fc polypeptide. Likewise, S267E/L328F defines an Fc variant with the substitutions S267E and L328F relative to the parent Fc polypeptide. The identity of the WT amino acid may be unspecified, in which case the aforementioned variant is referred to as 267E/328F. It is noted that the order in which substitutions are provided is arbitrary, that is to say that, for example, 267E/328F is the same Fc variant as 328F/267E, and so on. Unless otherwise noted, positions discussed herein are numbered according to the EU index as described in Kabat (Kabat et al., 1991, Sequences of Proteins of Immunological Interest, 5th Ed., United States Public Health Service, National Institutes of Health, Bethesda, hereby entirely incorporated by reference). Briefly, EU is the name of the first antibody molecule whose entire amino acid sequence was determined (Edelman et al., 1969, Proc Natl Acad Sci USA 63:78-85, hereby entirely incorporated by reference), and its amino acid sequence has become the standard numbering scheme for heavy chain constant regions. The EU protein has become the standard reference for defining numbering. Kabat et al. lists the EU sequence in a set of indices aligning it with other antibody sequences, serving as a necessary tool for aligning antibodies to the EU numbering scheme. Thus, as appreciated by those of skill in the art, the standard way of referencing the EU numbering is to refer to Kabat et al.'s alignment of sequences, because it puts EU in context with antibodies of other variable domain lengths. As such, as used herein, “according to the EU index,” “the EU index as in Kabat” or “numbering is according to the EU index, as in Kabat” refers to the numbering of the EU antibody as described in Kabat.

In certain embodiments, the Fc variants disclosed herein are based on human IgG sequences, and thus human IgG sequences are used as the “base” sequences against which other sequences are compared, including but not limited to sequences from other organisms, for example rodent and primate sequences. It is contemplated that, although the Fc variants disclosed herein are engineered in the context of one parent IgG, the variants may be engineered in or “transferred” to the context of another, second parent IgG. This is done by determining the “equivalent” or “corresponding” residues and substitutions between the first and second IgG, typically based on sequence or structural homology between the sequences of the first and second IgGs. In order to establish homology, the amino acid sequence of a first IgG outlined herein is directly compared to the sequence of a second IgG. After aligning the sequences, using one or more of the homology alignment programs known in the art (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first immunoglobulin are defined. Alignment of conserved residues may conserve 100% of such residues. However, alignment of greater than 75% or as little as 50% of conserved residues is also adequate to define equivalent residues. Equivalent residues may also be defined by determining structural homology between a first and second IgG that is at the level of tertiary structure for IgGs whose structures have been determined. In this case, equivalent residues are defined as those for which the atomic coordinates of two or more of the main chain atoms of a particular amino acid residue of the parent or precursor (N on N, CA on CA, Con C and O on O) are within about 0.13 nm, after alignment. In another embodiment, equivalent residues are within about 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent IgG in which the IgGs are made, what is meant to be conveyed is that the Fc variants discovered as disclosed herein may be engineered into any second parent IgG that has significant sequence or structural homology with the Fc variant. Thus, for example, if a variant antibody is generated wherein the parent antibody is human IgG1, by using the methods described above or other methods for determining equivalent residues, the variant antibody may be engineered in another IgG1 parent antibody that binds a different antigen, a human IgG2 parent antibody, a human IgA parent antibody, a mouse IgG2a or IgG2b parent antibody, and the like. Again, as described above, the context of the parent Fc variant does not affect the ability to transfer the Fc variants disclosed herein to other parent IgGs.

The term “greater affinity” or “improved affinity” or “enhanced affinity” or “better affinity” than a parent Fc polypeptide, as used herein is meant that an Fc variant binds to an Fc receptor with a significantly higher equilibrium constant of association (KA or Ka) or lower equilibrium constant of dissociation (KD or Kd) than the parent Fc polypeptide when the amounts of variant and parent polypeptide in the binding assay are essentially the same. For example, the Fc variant with improved Fc receptor binding affinity may display from 5 fold to 1000 fold, e.g. from 10 fold to 500 fold improvement in Fc receptor binding affinity compared to the parent Fc polypeptide, where Fc receptor binding affinity is determined, for example, by the binding methods disclosed herein, including but not limited to Biacore, by one skilled in the art. Accordingly, by “reduced affinity” as compared to a parent Fc polypeptide as used herein is meant that an Fc variant binds an Fc receptor with significantly lower KA or higher KD than the parent Fc polypeptide. Greater or reduced affinity can also be defined relative to an absolute level of affinity. For example, according to the data herein, WT (native) IgG1 binds FcγRIIb with an affinity of 1.5 mM, or 1500 nM. Furthermore, some Fc variants described herein bind FcγRIIb with an affinity 10-fold greater to WT IgG1. As disclosed herein, greater or enhanced affinity means having a KD lower than 100 nM, for example between 10 nM-100 nM, between 1-100 nM, or less than 1 nM.

In one embodiment, the Fc variants provide selectively enhanced affinity to FcγRIIb relative to one or more activating receptors. Selectively enhanced affinity means either that the Fc variant has improved affinity for FcγRIIb relative to the activating receptor(s) as compared to the parent Fc polypeptide but has reduced affinity for the activating receptor(s) as compared to the parent Fc polypeptide, or it means that the Fc variant has improved affinity for both FcγRIIb and activating receptor(s) as compared to the parent Fc polypeptide, however the improvement in affinity is greater for FcγRIIb than it is for the activating receptor(s). In alternate embodiments, the Fc variants reduce or ablate binding to one or more activating FcγRs, reduce or ablate binding to one or more complement proteins, reduce or ablate one or more FcγR-mediated effector functions, and/or reduce or ablate one or more complement-mediated effector functions.

The presence of different polymorphic forms of FcγRs provides yet another parameter that impacts the therapeutic utility of the Fc variants disclosed herein. Whereas the specificity and selectivity of a given Fc variant for the different classes of FcγRs significantly affects the capacity of an Fc variant to target a given antigen for treatment of a given disease, the specificity or selectivity of an Fc variant for different polymorphic forms of these receptors may in part determine which research or pre-clinical experiments may be appropriate for testing, and ultimately which patient populations may or may not respond to treatment. Thus, the specificity or selectivity of Fc variants disclosed herein to Fc receptor polymorphisms, including but not limited to FcγRIIa, FcγRIIIa, and the like, may be used to guide the selection of valid research and pre-clinical experiments, clinical trial design, patient selection, dosing dependence, and/or other aspects concerning clinical trials.

Fc variants disclosed herein may comprise modifications that modulate interaction with Fc receptors other than FcγRs, including but not limited to complement proteins, FcRn, and Fc receptor homologs (FcRHs). FcRHs include but are not limited to FcRFH, FcRH2, FcRH3, FcRH4, FcRH5, and FcRH6 (Davis et al., 2002, Immunol. Reviews 190:123-136). An important parameter that determines the most beneficial selectivity of a given Fc variant to treat a given disease is the context of the Fc variant. Thus, the Fc receptor selectivity or specificity of a given Fc variant will provide different properties depending on whether it composes an antibody, Fc fusion, or Fc variants with a coupled fusion partner. In one embodiment, an Fc receptor specificity of the Fc variant disclosed herein will determine its therapeutic utility. The utility of a given Fc variant for therapeutic purposes will depend on the epitope or form of the target antigen and the disease or indication being treated. For some targets and indications, greater FcγRIIb affinity and reduced activating FcγR-mediated effector functions may be beneficial. For other target antigens and therapeutic applications, it may be beneficial to increase affinity for FcγRIIb, or increase affinity for both FcγRIIb and activating receptors.

### Methods of Treating Relapsing Multiple Sclerosis

The present invention provides methods of treatment using an anti-CD19 antibodies (e.g., obexelimab, or a variant thereof). In some embodiments, a method comprises administering an anti-CD19 antibody (e.g., obexelimab, or a variant thereof) at a therapeutically effective dosing regimen.

In some embodiments, an anti-CD19 antibody (e.g., obexelimab, or a variant thereof) is administered at a therapeutically effective dosing regimen is administered via a subcutaneous, intravenous, intradermal, inhalation, transdermal, topical, transmucosal, intrathecal or rectal route of administration. In some embodiments, an anti-CD19 antibody (e.g., obexelimab, or variant thereof) is administered intravenously or subcutaneously. In some embodiments, an anti-CD19 antibody (e.g., obexelimab, or variant thereof) is administered intravenously. In some embodiments, an anti-CD19 antibody (e.g., obexelimab, or variant thereof) is administered subcutaneously.

In some embodiments, an anti-CD19 (e.g., obexelimab, or a variant thereof) is administered via subcutaneous (SC) injection. In some embodiments, an anti-CD19 (e.g., obexelimab, or a variant thereof) is administered via intravenous (IV) injection. In some embodiments, an anti-CD19 antibody (e.g., obexelimab, or a variant thereof) is administered via two SC injections. In some embodiments, an anti-CD19 antibody (e.g., obexelimab, or a variant thereof) is administered via a glass prefilled syringe.

### Dosing Regimens

In some embodiments, the present invention provides a method of treating MS comprising administering an anti-CD19 antibody (e.g., obexelimab, or a variant thereof) at a therapeutically effective dose.

In some embodiments, a therapeutically effective dosing regimen comprises administering an anti-CD19 antibody (e.g., obexelimab or a variant thereof), at a dose of 250 mg. In some embodiments, an anti-CD19 antibody (e.g., obexelimab, or a variant thereof) is administered at a therapeutically effective dose SC QW. In some embodiments, an anti-CD19 antibody (e.g., obexelimab, or a variant thereof) is administered at a dose of 250 mg SC every week (QW). In some embodiments, an anti-CD19 antibody (e.g., obexelimab, or a variant thereof) is administered at a therapeutically effective dose SC Q2W. In some embodiments, an anti-CD19 antibody (e.g., obexelimab, or a variant thereof) is administered at a dose of 250 mg SC Q2W. In some embodiments, an anti-CD19 antibody (e.g., obexelimab, or a variant thereof) is administered at a dose of 250 mg SC Q3W. In some embodiments, an anti-CD19 antibody (e.g., obexelimab, or a variant thereof) is administered at a therapeutically effective dose SC Q3W. In some embodiments, an anti-CD19 antibody (e.g., obexelimab, or a variant thereof) is administered at a dose of 250 mg SC Q4W. In some embodiments, an anti-CD19 antibody (e.g., obexelimab, or a variant thereof) is administered at a therapeutically effective dose SC Q4W. In some embodiments, an anti-CD19 antibody (e.g., obexelimab, or a variant thereof) is administered at a therapeutically effective dosing regimen sufficient to achieve a Cmax of 14 to 25 μg/mL. In some embodiments, anti-CD19 antibody (e.g., obexelimab, or a variant thereof) is administered at a therapeutically effective dosing regimen sufficient to achieve an AUC of 3000 to 7400 (μg/mL·h). In some embodiments, an anti-CD19 antibody (e.g., obexelimab, or a variant thereof) is administered a therapeutically effective dosing regimen as described in Table 3.

TABLE 3
Exemplary Dosing Regimens

| | Cmax | AUC |
|---|---|---|
| Dosage regimen | (μg/mL) | (μg/mL · h) |
| 250 mg SC QW | 24.8 | 7375c |
| 250 mg SC Q2W | 14.7 | 3095 |

### Duration of Treatment

In some embodiments, a method of treating MS, or a form thereof (e.g., RMS), comprises therapeutically administering an anti-CD19 antibody (e.g., obexelimab or a variant thereof) for a duration of time. In some embodiments, an anti-CD19 antibody (e.g., obexelimab, or a variant thereof) is administered at a therapeutically effective dosing regimen for 2-76 weeks. In some embodiments, an anti-CD19 antibody (e.g., obexelimab, or a variant thereof) is administered at a therapeutically effective dosing regimen for 2-52 weeks. In some embodiments, an anti-CD19 antibody (e.g., obexelimab, or a variant thereof) is administered at a therapeutically effective dosing regimen for 2-24 weeks. In some embodiments, an anti-CD19 antibody (e.g., obexelimab, or a variant thereof) is administered at a therapeutically effective dosing regimen for 2 weeks. In some embodiments, an anti-CD19 antibody (e.g., obexelimab, or a variant thereof) is administered at a therapeutically effective dosing regimen for 3 weeks. In some embodiments, an anti-CD19 antibody (e.g., obexelimab, or a variant thereof) is administered at a therapeutically effective dosing regimen for 4 weeks. In some embodiments, an anti-CD19 antibody (e.g., obexelimab, or a variant thereof) is administered at a therapeutically effective dosing regimen for 5 weeks. In some embodiments an anti-CD19 antibody (e.g., obexelimab, or a variant thereof) is administered at a therapeutically effective dosing regimen for 6 weeks. In some embodiments, an anti-CD19 antibody (e.g., obexelimab, or a variant thereof) is administered at a therapeutically effective dosing regimen for 7 weeks. In some embodiments, an anti-CD19 antibody (e.g., obexelimab, or a variant thereof) is administered at a therapeutically effective dosing regimen for 8 weeks. In some embodiments, an anti-CD19 antibody (e.g., obexelimab, or a variant thereof) is administered at a therapeutically effective dosing regimen for 9 weeks. In some embodiments, an anti-CD19 antibody (e.g., obexelimab, or a variant thereof) is administered at a therapeutically effective dosing regimen for 10 weeks. In some embodiments, an anti-CD19 antibody (e.g., obexelimab, or a variant thereof) is administered at a therapeutically effective dosing regimen for 11 weeks. In some embodiments, an anti-CD19 antibody (e.g., obexelimab, or a variant thereof) is administered at a therapeutically effective dosing regimen for 12 weeks. In some embodiments, an anti-CD19 antibody (e.g., obexelimab, or a variant thereof) is administered at a therapeutically effective dosing regimen for 13 weeks. In some embodiments, an anti-CD19 antibody (e.g., obexelimab, or a variant thereof) is administered at a therapeutically effective dosing regimen for 14 weeks. In some embodiments, an anti-CD19 antibody (e.g., obexelimab, or a variant thereof) is administered at a therapeutically effective dosing regimen for 15 weeks. In some embodiments, an anti-CD19 antibody (e.g., obexelimab, or a variant thereof) is administered at a therapeutically effective dosing regimen for 16 weeks. In some embodiments, an anti-CD19 antibody (e.g., obexelimab, or a variant thereof) is administered at a therapeutically effective dosing regimen for 17 weeks. In some embodiments the dose of an anti-CD19 antibody (e.g., obexelimab, or a variant thereof) is administered at a therapeutically effective dosing regimen for 18 weeks. In some embodiments an anti-CD19 antibody (e.g., obexelimab, or a variant thereof) is administered at a therapeutically effective dosing regimen for 19 weeks. In some embodiments, an anti-CD19 antibody (e.g., obexelimab, or a variant thereof) is administered at a therapeutically effective dosing regimen for 20 weeks. In some embodiments, an anti-CD19 antibody (e.g., obexelimab, or a variant thereof) is administered at a therapeutically effective dosing regimen for 21 weeks. In some embodiments, an anti-CD19 antibody (e.g., obexelimab, or a variant thereof) is administered at a therapeutically effective dosing regimen for 22 weeks. In some embodiments, an anti-CD19 antibody (e.g., obexelimab, or a variant thereof) is administered at a therapeutically effective dosing regimen for 23 weeks. In some embodiments, an anti-CD19 antibody (e.g., obexelimab or a variant thereof) is administered at a therapeutically effective dosing regimen for 24 weeks. In some embodiments, an anti-CD19 antibody (e.g., obexelimab or a variant thereof) is administered at a therapeutically effective dosing regimen for 28 weeks. In some embodiments, an anti-CD19 antibody (e.g., obexelimab or a variant thereof) is administered at a therapeutically effective dosing regimen for 32 weeks. In some embodiments, an anti-CD19 antibody (e.g., obexelimab or a variant thereof) is administered at a therapeutically effective dosing regimen for 36 weeks. In some embodiments, an anti-CD19 antibody (e.g., obexelimab or a variant thereof) is administered at a therapeutically effective dosing regimen for 40 weeks. In some embodiments, an anti-CD19 antibody (e.g., obexelimab or a variant thereof) is administered at a therapeutically effective dosing regimen for 44 weeks. In some embodiments, an anti-CD19 antibody (e.g., obexelimab or a variant thereof) is administered at a therapeutically effective dosing regimen for 48 weeks. In some embodiments, an anti-CD19 antibody (e.g., obexelimab or a variant thereof) is administered at a therapeutically effective dosing regimen for 52 weeks. In some embodiments, an anti-CD19 antibody (e.g., obexelimab or a variant thereof) is administered at a therapeutically effective dosing regimen for 56 weeks. In some embodiments, an anti-CD19 antibody (e.g., obexelimab or a variant thereof) is administered at a therapeutically effective dosing regimen for 60 weeks. In some embodiments, an anti-CD19 antibody (e.g., obexelimab or a variant thereof) is administered at a therapeutically effective dosing regimen for 64 weeks. In some embodiments, an anti-CD19 antibody (e.g., obexelimab or a variant thereof) is administered at a therapeutically effective dosing regimen for 68 weeks. In some embodiments, an anti-CD19 antibody (e.g., obexelimab or a variant thereof) is administered at a therapeutically effective dosing regimen for 72 weeks. In some embodiments, an anti-CD19 antibody (e.g., obexelimab or a variant thereof) is administered at a therapeutically effective dosing regimen for 76 weeks.

In some embodiments, an equivalent dosing regimen comprises administering an anti-CD19 antibody at a dose at an administration interval sufficient to reduce one or more symptoms associated with MS. For example, an equivalent dosing regimen of 250 mg every week is 125 mg twice a week, 500 mg every two weeks, 750 mg every three weeks, 1000 mg every four weeks.

### Patient Outcomes

Patient outcomes can be determined by several factors. In some embodiments, the number of new and/or enlarged GdE T1 hyperintense lesions can be measured by brain MRI during the course of treatment with an anti-CD19 antibody (e.g., obexelimab or a variant thereof). In some embodiments, administering an anti-CD19 antibody (e.g., obexelimab or a variant thereof) at a therapeutically effective dosing regimen is sufficient to stabilize the number of GdE T1 hyperintense lesions as compared to pre-treatment status. In some embodiments, administering an anti-CD19 antibody (e.g., obexelimab or a variant thereof) at a therapeutically effective dosing regimen is sufficient to reduce the number of new GdE T1 hyperintense lesions as compared to one or more control individuals with similar disease without treatment. In some embodiments, administering an anti-CD19 antibody (e.g., obexelimab or a variant thereof) at a therapeutically effective dosing regimen is sufficient to reduce the volume of new or enlarged GdE T1 hyperintense lesions as compared to one or more control individuals with similar disease without treatment.

In some embodiments the number of new and/or enlarged T2 hyperintense lesions can be measured by brain MRI during the course of treatment with an anti-CD19 antibody (e.g., obexelimab or a variant thereof). In some embodiments, administering an anti-CD19 antibody (e.g., obexelimab or a variant thereof) at a therapeutically effective dosing regimen is sufficient to stabilize the number of T2 hyperintense lesions as compared to pre-treatment status. In some embodiments, administering an anti-CD19 antibody (e.g., obexelimab or a variant thereof) at a therapeutically effective dosing regimen is sufficient to reduce the number of new T2 hyperintense lesions as compared to one or more control individuals with similar disease without treatment. In some embodiments, administering an anti-CD19 antibody (e.g., obexelimab or a variant thereof) at a therapeutically effective dosing regimen is sufficient to reduce the volume of new or enlarged T2 hyperintense lesions as compared to one or more control individuals with similar disease without treatment.

In some embodiments, a change in the number of rim lesions can be measured by brain MRI during the course of treatment with an anti-CD19 antibody. In some embodiments, administering an anti-CD19 antibody (e.g., obexelimab or a variant thereof) at a therapeutically effective dosing regimen is sufficient to stabilize the number of rim lesions as compared to pre-treatment status. In some embodiments, administering an anti-CD19 antibody (e.g., obexelimab or a variant thereof) at a therapeutically effective dosing regimen is sufficient to reduce the number of new rim lesions as compared to one or more control individuals with similar disease without treatment. In some embodiments, administering an anti-CD19 antibody (e.g., obexelimab or a variant thereof) is sufficient to reduce the conversion from T1 to new phase rim lesions as compared to one or more control individuals with similar disease without treatment.

In some embodiments, a change in brain volume can be measured by MRI during the course of treatment with an anti-CD19 antibody. In some embodiments, administering an anti-CD19 antibody (e.g., obexelimab or a variant thereof) at a therapeutically effective dosing regimen is sufficient to preserve brain volume as compared to one or more control individuals with similar disease without treatment. In some embodiments, administering an anti-CD19 antibody (e.g., obexelimab or a variant thereof) is sufficient to preserve white matter volume as compared to one or more control individuals with similar disease without treatment. In some embodiments, administering an anti-CD19 antibody (e.g., obexelimab or a variant thereof) is sufficient to preserve grey matter volume as compared to one or more control individuals with similar disease without treatment. In some embodiments, administering an anti-CD19 antibody (e.g., obexelimab or a variant thereof) is sufficient to preserve brain volume in the cerebellum as compared to one or more control individuals with similar disease without treatment. In some embodiments, administering an anti-CD19 antibody (e.g., obexelimab or a variant thereof) is sufficient to preserve brain volume in the cerebellum as compared to one or more control individuals with similar disease without treatment. In some embodiments, administering an anti-CD19 antibody (e.g., obexelimab or a variant thereof) is sufficient to preserve brain volume in the hippocampus as compared to one or more control individuals with similar disease without treatment. In some embodiments, administering an anti-CD19 antibody (e.g., obexelimab or a variant thereof) is sufficient to preserve brain volume in the frontal lobes as compared to one or more control individuals with similar disease without treatment. In some embodiments, administering an anti-CD19 antibody (e.g., obexelimab or a variant thereof) is sufficient to preserve brain volume in the parietal lobe as compared to one or more control individuals with similar disease without treatment. In some embodiments, administering an anti-CD19 antibody (e.g., obexelimab or a variant thereof) is sufficient to preserve brain volume in the temporal lobe as compared to one or more control individuals with similar disease without treatment. In some embodiments, administering an anti-CD19 antibody (e.g., obexelimab or a variant thereof) is sufficient to preserve brain volume in the occipital lobe as compared to one or more control individuals with similar disease without treatment. In some embodiments, administering an anti-CD19 antibody (e.g., obexelimab or a variant thereof) is sufficient to preserve cortical gray matter as compared to one or more control individuals with similar disease without treatment. In some embodiments, administering an anti-CD19 antibody (e.g., obexelimab or a variant thereof) is sufficient to preserve brain volume in the spinal cord as compared to one or more control individuals with similar disease without treatment. In some embodiments, administering an anti-CD19 antibody (e.g., obexelimab or a variant thereof) is sufficient to preserve brain volume in the optic nerve as compared to one or more control individuals with similar disease without treatment. In some embodiments, administering an anti-CD19 antibody (e.g., obexelimab or a variant thereof) is sufficient to preserve brain volume in the thalamus as compared to one or more control individuals with similar disease without treatment. In some embodiments, administering an anti-CD19 antibody (e.g., obexelimab or a variant thereof) is sufficient to preserve brain volume in the cerebellum as compared to one or more control individuals with similar disease without treatment. In some embodiments, administering an anti-CD19 antibody (e.g., obexelimab or a variant thereof) is sufficient to preserve brain volume in the cerebral cortex as compared to one or more control individuals with similar disease without treatment.

In some embodiments, localized atrophy in the brain can be measured by MRI during the course of treatment with an anti-CD19 antibody. In some embodiments, administering an anti-CD19 antibody (e.g., obexelimab or a variant thereof) at a therapeutically effective dosing regimen is sufficient to prevent localized atrophy in parts of the brain as compared to one or more control individuals with similar disease without treatment. In some embodiments, administering an anti-CD19 antibody (e.g., obexelimab or a variant thereof) is sufficient to prevent localized atrophy in the cerebellum as compared to one or more control individuals with similar disease without treatment. In some embodiments, administering an anti-CD19 antibody (e.g., obexelimab or a variant thereof) is sufficient to prevent localized atrophy in the frontal lobes as compared to one or more control individuals with similar disease without treatment. In some embodiments, administering an anti-CD19 antibody (e.g., obexelimab or a variant thereof) is sufficient to prevent localized atrophy in the parietal lobe as compared to one or more control individuals with similar disease without treatment. In some embodiments, administering an anti-CD19 antibody (e.g., obexelimab or a variant thereof) is sufficient to prevent localized atrophy in the temporal lobe as compared to one or more control individuals with similar disease without treatment. In some embodiments, administering an anti-CD19 antibody (e.g., obexelimab or a variant thereof) is sufficient to prevent localized atrophy in the occipital lobe as compared to one or more control individuals with similar disease without treatment. In some embodiments, administering an anti-CD19 antibody (e.g., obexelimab or a variant thereof) is sufficient to prevent localized atrophy in the hippocampus as compared to one or more control individuals with similar disease without treatment. In some embodiments, administering an anti-CD19 antibody (e.g., obexelimab or a variant thereof) is sufficient to prevent localized atrophy in the cerebellum as compared to one or more control individuals with similar disease without treatment. In some embodiments, administering an anti-CD19 antibody (e.g., obexelimab or a variant thereof) is sufficient to prevent localized atrophy in the optic nerve as compared to one or more control individuals with similar disease without treatment. In some embodiments, administering an anti-CD19 antibody (e.g., obexelimab or a variant thereof) is sufficient to prevent localized atrophy in the thalamus as compared to one or more control individuals with similar disease without treatment. In some embodiments, localized atrophy in the brain can be measured by MRI during the course of treatment with an anti-CD19 antibody. In some embodiments, administering an anti-CD19 antibody (e.g., obexelimab or a variant thereof) is sufficient to prevent localized atrophy in cortical gray matter as compared to one or more control individuals with similar disease without treatment.

In some embodiments, serum cytokines, chemokines, and neurology markers can be measured during the course of treatment with an anti-CD19 antibody. In some embodiments, administering an anti-CD19 antibody (e.g., obexelimab or a variant thereof) at a therapeutically effective dosing regimen is sufficient to reduce serum concentrations of cytokines. In some embodiments the course of treatment with an anti-CD19 antibody (e.g., obexelimab or a variant thereof) at a therapeutically effective dosing regimen is sufficient to reduce serum concentrations of chemokines. In some embodiments, administering an anti-CD19 antibody (e.g., obexelimab or a variant thereof) at a therapeutically effective dosing regimen is sufficient to reduce serum concentrations of NfL. In some embodiments, administering an anti-CD19 antibody (e.g., obexelimab or a variant thereof) at a therapeutically effective dosing regimen is sufficient to reduce serum concentrations of Tau. In some embodiments, administering an anti-CD19 antibody (e.g., obexelimab or a variant thereof) at a therapeutically effective dosing regimen is sufficient to reduce serum concentrations of GFAP. In some embodiments, administering an anti-CD19 antibody (e.g., obexelimab or a variant thereof) at a therapeutically effective dosing regimen is sufficient to reduce serum concentrations of CHI3L1. In some embodiments, administering an anti-CD19 antibody (e.g., obexelimab or a variant thereof) at a therapeutically effective dosing regimen is sufficient to reduce serum concentrations of CXCL-13. In some embodiments, administering an anti-CD19 antibody (e.g., obexelimab or a variant thereof) at a therapeutically effective dosing regimen is sufficient to reduce serum concentrations of UCH-L1. In some embodiments, administering an anti-CD19 antibody (e.g., obexelimab or a variant thereof) at a therapeutically effective dosing regimen is sufficient to reduce serum concentrations of TREM2.

In some embodiments, administering an anti-CD19 antibody (e.g., obexelimab or a variant thereof) at a therapeutically effective dosing regimen is sufficient to reduce the number or annualized rate of relapses.

In some embodiments, patient-reported outcomes (PROs) are used to assess therapeutic efficacy in treating MS. In some embodiments, Patient Global Impression of Severity (PGIS)-Fatigue, Patient Global Impression of Change (PGIC)-Fatigue, Multiple Sclerosis Impact Scale (MSIS)-29, Patient Reported Outcome Measuring System (PROMIS)-Fatigue-MS-8, or other similar tests are used to assess therapeutic efficacy in treating MS. In some embodiments, administering an anti-CD19 antibody (e.g., obexelimab or a variant thereof) at a therapeutically efficient dosing regimen is sufficient to reduce fatigue experienced by a patient. In some embodiments, administering an anti-CD19 antibody (e.g., obexelimab or a variant thereof) at a therapeutically effective dosing regimen is sufficient to stabilize a patient's PGIS-Fatigue score as compared to baseline. In some embodiments, administering an anti-CD19 antibody (e.g., obexelimab or a variant thereof) at a therapeutically effective dosing regimen is sufficient to improve a patient's PGIS-Fatigue score as compared to baseline. In some embodiments, administering an anti-CD19 antibody (e.g., obexelimab or a variant thereof) at a therapeutically effective dosing regimen is sufficient to stabilize a patient's PGIC-Fatigue score as compared to baseline. In some embodiments, administering an anti-CD19 antibody (e.g., obexelimab or a variant thereof) at a therapeutically effective dosing regimen is sufficient to improve a patient's PGIC-Fatigue score as compared to baseline. In some embodiments, administering an anti-CD19 antibody (e.g., obexelimab or a variant thereof) at a therapeutically effective dosing regimen is sufficient to stabilize a patient's MSIS-29 score as compared to baseline. In some embodiments, administering an anti-CD19 antibody (e.g., obexelimab or a variant thereof) at a therapeutically effective dosing regimen is sufficient to improve a patient's MSIS-29 score as compared to baseline. In some embodiments, administering an anti-CD19 antibody (e.g., obexelimab or a variant thereof) at a therapeutically effective dosing regimen is sufficient to stabilize a patient's PROMIS-Fatigue-MS-8 score as compared to baseline. In some embodiments, administering an anti-CD19 antibody (e.g., obexelimab or a variant thereof) at a therapeutically effective dosing regimen is sufficient to improve a patient's PROMIS-Fatigue-MS-8 score as compared to baseline. In some embodiments, disability is measured by a disability test, such as for example, EDSS. In some embodiments, administering an anti-CD19 antibody (e.g., obexelimab or a variant thereof) at a therapeutically effective dose is sufficient to stabilize a patient's EDSS score as compared to pre-treatment status. In some embodiments, administering an anti-CD19 antibody (e.g., obexelimab or a variant thereof) at a therapeutically effective dose is sufficient to reduce a patient's EDSS score as compared to pre-treatment status. In some embodiments, administering an anti-CD19 antibody (e.g., obexelimab or a variant thereof) at a therapeutically effective dose is sufficient to slow disease progression as compared to one or more control individuals with similar disease without treatment, wherein disease progression is measured by an increase in a patient's EDSS score.

In some embodiments, a patient's annualized relapse rate (ARR) is used to assess therapeutic efficacy in treating MS. In some embodiments, a relapse occurs when a patient experiences new, worsening, or recurrent neurological symptoms attributed to MS that last for at least 24 hours without fever or infection or adverse reaction to prescribed medication, preceded by a stable or improving neurological status of at least 30 days. In some embodiments, a relapse occurs when a patient's EDSS score increases by at least 0.5 points. In some embodiments, administering an anti-CD19 antibody (e.g., obexelimab, or a variant thereof) is sufficient to reduce a patient's ARR as compared to pre-treatment status. In some embodiments, administering an anti-CD19 antibody (e.g., obexelimab, or a variant thereof) is sufficient to reduce a patient's ARR as compared to one or more control individuals with similar disease without treatment. In some embodiments, administering an anti-CD19 antibody (e.g., obexelimab, or a variant thereof) is sufficient to reduce a patient's ARR to below 0.15. In some embodiments, administering an anti-CD19 antibody (e.g., obexelimab, or a variant thereof) is sufficient to reduce a patient's ARR to below 0.14. In some embodiments, administering an anti-CD19 antibody (e.g., obexelimab, or a variant thereof) is sufficient to reduce a patient's ARR to below 0.13. In some embodiments, administering an anti-CD19 antibody (e.g., obexelimab, or a variant thereof) is sufficient to reduce a patient's ARR to below 0.12. In some embodiments, administering an anti-CD19 antibody (e.g., obexelimab, or a variant thereof) is sufficient to reduce a patient's ARR to below 0.11. In some embodiments, administering an anti-CD19 antibody (e.g., obexelimab, or a variant thereof) is sufficient to reduce a patient's ARR to below 0.10. In some embodiments, administering an anti-CD19 antibody (e.g., obexelimab, or a variant thereof) is sufficient to reduce a patient's ARR to below 0.09. In some embodiments, administering an anti-CD19 antibody (e.g., obexelimab, or a variant thereof) is sufficient to reduce a patient's ARR to below 0.08. In some embodiments, administering an anti-CD19 antibody (e.g., obexelimab, or a variant thereof) is sufficient to reduce a patient's ARR to below 0.07. In some embodiments, administering an anti-CD19 antibody (e.g., obexelimab, or a variant thereof) is sufficient to reduce a patient's ARR to below 0.06. In some embodiments, administering an anti-CD19 antibody (e.g., obexelimab, or a variant thereof) is sufficient to reduce a patient's ARR to below 0.05. In some embodiments, administering an anti-CD19 antibody (e.g., obexelimab, or a variant thereof) is sufficient to reduce a patient's ARR to below 0.04. In some embodiments, administering an anti-CD19 antibody (e.g., obexelimab, or a variant thereof) is sufficient to reduce a patient's ARR to below 0.03. In some embodiments, administering an anti-CD19 antibody (e.g., obexelimab, or a variant thereof) is sufficient to reduce a patient's ARR to below 0.02. In some embodiments, administering an anti-CD19 antibody (e.g., obexelimab, or a variant thereof) is sufficient to reduce a patient's ARR to below 0.01. In some embodiments, administering an anti-CD19 antibody (e.g., obexelimab, or a variant thereof) is sufficient to substantially prevent the occurrence of relapse.

In some embodiments, therapeutic efficacy is assessed by confirmed disability progression. In some embodiments, administering an anti-CD19 antibody (e.g., obexelimab, or a variant thereof) is sufficient to prevent confirmed disability progression. In some embodiments, administering an anti-CD19 antibody (e.g., obexelimab, or a variant thereof) is sufficient to delay the onset of confirmed disability progression as compared to one or more control individuals with similar disease without treatment. In some embodiments, confirmed disability progression is assessed using a 9-hole peg test (9HPT). In some embodiments, confirmed disability progression is assessed as an increase of ≥20% from a patient's baseline time in the 9HPT. In some embodiments, administering an anti-CD19 antibody (e.g., obexelimab, or a variant thereof) is sufficient to prevent an increase of ≥20% from a patient's baseline time in a 9HPT. In some embodiments, administering an anti-CD19 antibody (e.g., obexelimab, or a variant thereof) is sufficient to delay the onset of an increase of ≥20% from a patient's baseline time in a 9HPT. In some embodiments, confirmed disability progression is assessed using a Timed 25-foot walk (T25FW) test. In some embodiments, confirmed disability progression is assessed as an increase of ≥20% from a patient's baseline time in a T25FW test. In some embodiments, administering an anti-CD19 antibody (e.g., obexelimab, or a variant thereof) is sufficient to prevent an increase of ≥20% from a patient's baseline time in a T25FW test. In some embodiments, administering an anti-CD19 antibody (e.g., obexelimab, or a variant thereof) is sufficient to delay the onset of an increase of ≥20% from a patient's baseline time in a T25FW test. In some embodiments, confirmed disability progression is assessed using change in EDSS score. In some embodiments, confirmed disability progression is assessed as an increase of ≥1.5 EDSS score in patients with a baseline of 0. In some embodiments, administering an anti-CD19 antibody (e.g., obexelimab, or a variant thereof) is sufficient to prevent an increase of ≥1.5 EDSS score in patients with a baseline of 0. In some embodiments, administering an anti-CD19 antibody (e.g., obexelimab, or a variant thereof) is sufficient to delay the onset of an increase of ≥1.5 EDSS score in patients with a baseline of 0. In some embodiments, confirmed disability progression is assessed as an increase of ≥1.0 EDSS score in patients with a baseline of 0.5 to 5.0. In some embodiments, administering an anti-CD19 antibody (e.g., obexelimab, or a variant thereof) is sufficient to prevent an increase of ≥1.0 EDSS score in patients with a baseline of 0.5 to 5.0. In some embodiments, administering an anti-CD19 antibody (e.g., obexelimab, or a variant thereof) is sufficient to delay the onset of an increase of ≥1.0 EDSS score in patients with a baseline of 0.5 to 5.0. In some embodiments, confirmed disability progression is assessed as an increase of ≥1.0 EDSS score in patients with a baseline of <5.0. In some embodiments, administering an anti-CD19 antibody (e.g., obexelimab, or a variant thereof) is sufficient to prevent an increase of ≥1.0 EDSS score in patients with a baseline of <5.0. In some embodiments, administering an anti-CD19 antibody (e.g., obexelimab, or a variant thereof) is sufficient to delay the onset of an increase of ≥1.0 EDSS score in patients with a baseline of <5.0. In some embodiments, confirmed disability progression is assessed as an increase of ≥0.5 EDSS score in patients with a baseline of ≥5.5. In some embodiments, administering an anti-CD19 antibody (e.g., obexelimab, or a variant thereof) is sufficient to prevent an increase of ≥0.5 EDSS score in patients with a baseline of ≥5.5. In some embodiments, administering an anti-CD19 antibody (e.g., obexelimab, or a variant thereof) is sufficient to delay the onset of an increase of ≥0.5 EDSS score in patients with a baseline of ≥5.5.

### Formulation and Pharmaceutical Compositions

The present invention provides pharmaceutical compositions and formulations of anti-CD19 antibodies (e.g., obexelimab). Formulations of the anti-CD19 antibody disclosed herein are prepared for storage by mixing said antibody having the desired degree of purity with optional pharmaceutically acceptable carriers, excipients or stabilizers (Remington's Pharmaceutical Sciences 16th edition, Osol, A. Ed., 1980, incorporated entirely by reference), in the form of lyophilized formulations or aqueous solutions.

In some embodiments, pharmaceutical compositions of interest comprise an anti-CD19 antibody (e.g., obexelimab) at various concentrations. In some embodiments, suitable formulations may comprise the antibody of interest at a concentration up to 250 mg/mL (e.g., up to 225 mg/mL, up to 200 mg/mL, up to 150 mg/mL, up to 140 mg/mL, up to 130 mg/mL, up to 125 mg/mL, up to 120 mg/mL, up to 115 mg/mL, up to 110 mg/mL, up to 105 mg/mL, up to 100 mg/mL, up to 90 mg/mL, up to 80 mg/mL, up to 70 mg/mL, up to 60 mg/mL, up to 50 mg/mL, up to 40 mg/mL, up to 30 mg/mL, up to 25 mg/mL, up to 20 mg/mL, up to 10 mg/mL).

In some embodiments, suitable formulations may contain the anti-CD19 antibody at a concentration ranging between 10-300 mg/mL (e.g., 10-250 mg/mL, 10-200 mg/mL, 10-180 mg/mL, 10-160 mg/mL, 10-150 mg/mL, 10-140 mg/mL, 10-130 mg/mL, 10-125 mg/mL, 100-125 mg/mL, 100-180 mg/mL, 100-150 mg/mL, 100-130 mg/mL, 100-125 mg/mL, 100-170 mg/mL, 100-160 mg/mL, 100-150 mg/mL, 100-200 mg/mL, or 120-130 mg/mL).

In some embodiments, a formulation suitable for SC administration contains a protein of interest at a concentration of approximately 100 mg/mL, 115 mg/mL, 120 mg/mL, 125 mg/mL, 130 mg/mL, 135 mg/mL, 140 mg/mL, 145 mg/mL, 150 mg/mL, 200 mg/mL, or 300 mg/mL.

In some embodiments, isotonic solutions are used. In some embodiments, slightly hypertonic solutions (e.g., up to 300 mM (e.g., up to 250 mM, 200 mM, 175 mM, 150 mM, 125 mM) sodium chloride in 5 mM sodium phosphate at pH 7.0) and sugar-containing solutions (e.g., up to 3% (e.g., up to 2.4%, 2.0%, 1.5%, 1.0%) sucrose in 5 mM sodium phosphate at pH 7.0). In some embodiments, a suitable formulation composition is saline (e.g., 150 mM NaCl in water).

Many therapeutic agents, and in particular the antibodies of the present invention, require controlled pH and specific excipients to maintain their solubility and stability in the pharmaceutical compositions of the present invention.

The pH of the pharmaceutical composition is an additional factor which is capable of altering the solubility of an anti-CD19 antibody (e.g., obexelimab) in an aqueous pharmaceutical composition. In some embodiments, pharmaceutical compositions of the present invention contain one or more buffers. In some embodiments, compositions according to the invention contain an amount of buffer sufficient to maintain the optimal pH of said composition, e.g., between 4.0-8.0, between 5.0-7.5, between 5.5-7.0, between 6.0-7.0 or between 6.0-7.5. In other embodiments, the buffer comprises up to 50 mM (e.g., up to 45 mM, 40 mM, 35 mM, 30 mM, 25 mM, 20 mM, 15 mM, 10 mM, 5 mM) of sodium phosphate. Suitable buffers include, for example acetate, succinate, citrate, phosphate, other organic acids and tris (hydroxymethyl) aminomethane (“Tris”).

Suitable buffer concentrations can be from 1 mM to 100 mM, or from 3 mM to 20 mM, depending, for example, on the buffer and the desired isotonicity of the formulation. In some embodiments, a suitable buffering agent is present at a concentration of approximately 1 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, formulations contain an isotonicity agent to keep the formulations isotonic. Exemplary isotonicity agents include, but are not limited to, glycine, sorbitol, mannitol, sodium chloride and arginine. In some embodiments, suitable isotonic agents may be present in formulations at a concentration from 0.01-5% (e.g., 0.05, 0.1, 0.15, 0.2, 0.3, 0.4, 0.5, 0.75, 1.0, 1.25, 1.5, 2.0, 2.5, 3.0, 4.0 or 5.0%) by weight.

In some embodiments, formulations may contain a stabilizing agent to protect the antibody. Typically, a suitable stabilizing agent is a non-reducing sugar such as sucrose, raffinose, trehalose, or amino acids such as glycine, arginine and methionine. The amount of stabilizing agent in a formulation is generally such that the formulation will be isotonic. However, hypertonic formulations may also be suitable. In addition, the amount of stabilizing agent must not be too low such that an unacceptable amount of degradation/aggregation of the antibody occurs. Exemplary stabilizing agent concentrations in the formulation may range from 1 mM to 400 mM (e.g., from 30 mM to 300 mM, and from 50 mM to 100 mM), or alternatively, from 0.1% to 15% (e.g., from 1% to 10%, from 5% to 15%, from 5% to 10%) by weight. In some embodiments, the ratio of the mass amount of the stabilizing agent and the therapeutic agent is 1:1. In other embodiments, the ratio of the mass amount of the stabilizing agent and the therapeutic agent can be 0.1:1, 0.2:1, 0.25:1, 0.4:1, 0.5:1, 1:1, 2:1, 2.6:1, 3:1, 4:1, 5:1, 10:1, or 20:1. In some embodiments, suitable for lyophilization, the stabilizing agent is also a lyoprotectants.

The pharmaceutical compositions, formulations and related methods of the invention are useful for delivering anti-CD19 antibodies (e.g., subcutaneously) and for the treatment of the associated diseases. The pharmaceutical compositions of the present invention are particularly useful for delivering anti-CD19 antibodies (e.g., obexelimab) to patients suffering from RMS.

In some embodiments, it is desirable to add a surfactant to formulations. Exemplary surfactants include nonionic surfactants such as Polysorbates (e.g., Polysorbates 20 or 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristarnidopropyl-, palmidopropyl-, or isostearamidopropyl-dimethylamine; sodium methyl cocoyl-, or disodium methyl ofeyl-taurate; and the MONAQUAT™ series (Mona Industries, Inc., Paterson, N.J.), polyethyl glycol, polypropyl glycol, and copolymers of ethylene and propylene glycol (e.g., Pluronics, PF68, etc). Typically, the amount of surfactant added is such that it reduces aggregation of the protein and minimizes the formation of particulates or effervescences. For example, a surfactant may be present in a formulation at a concentration from 0.001-0.5% (e.g., 0.005-0.05%, or 0.005-0.01%). In particular, a surfactant may be present in a formulation at a concentration of approximately 0.005%, 0.01%, 0.02%, 0.1%, 0.2%, 0.3%, 0.4%, or 0.5%, etc.

In some embodiments, suitable formulations may further include one or more bulking agents, in particular, for lyophilized formylations. A “bulking agent” is a compound which adds mass to the lyophilized mixture and contributes to the physical structure of the lyophilized cake. For example, a bulking agent may improve the appearance of lyophilized cake (e.g., essentially uniform lyophilized cake). Suitable bulking agents include, but are not limited to, sodium chloride, lactose, mannitol, glycine, sucrose, trehalose, hydroxyethyl starch. Exemplary concentrations of bulking agents are from 1% to 10% (e.g., 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, and 10.0%).

Formulations in accordance with the present invention can be assessed based on product quality analysis, reconstitution time (if lyophilized), quality of reconstitution (if lyophilized), high molecular weight, moisture, and glass transition temperature. Typically, protein quality and product analysis include product degradation rate analysis using methods including, but not limited to, size exclusion HPLC (SE-HPLC), cation exchange-HPLC (CEX-HPLC), X-ray diffraction (XRD), modulated differential scanning calorimetry (mDSC), reversed phase HPLC (RP-HPLC), multi-angle light scattering (MALS), fluorescence, ultraviolet absorption, nephelometry, capillary electrophoresis (CE), SDS-PAGE, and combinations thereof. In some embodiments, evaluation of product in accordance with the present invention may include a step of evaluating appearance (either liquid or cake appearance).

Generally, formulations (lyophilized or aqueous) can be stored for extended periods of time at room temperature. Storage temperature may typically range from 0° C. to 45° C. (e.g., 4° C., 20° C., 25° C., 45° C. etc.). Formulations may be stored for a period of months to a period of years. Storage time generally will be 24 months, 12 months, 6 months, 4.5 months, 3 months, 2 months or 1 month. Formulations can be stored directly in the container used for administration, eliminating transfer steps.

Formulations can be stored directly in the lyophilization container (if lyophilized), which may also function as the reconstitution vessel, eliminating transfer steps. Alternatively, lyophilized product formulations may be measured into smaller increments for storage. Storage should generally avoid circumstances that lead to degradation of the proteins, including but not limited to exposure to sunlight, UV radiation, other forms of electromagnetic radiation, excessive heat or cold, rapid thermal shock, and mechanical shock.

In some embodiments, formulations according to the present invention are in a liquid or aqueous form. In some embodiments, formulations of the present invention are lyophilized. Such lyophilized formulations may be reconstituted by adding one or more diluents thereto prior to administration to a patient. Suitable diluents include, but are not limited to, sterile water, bacteriostatic water for injection and sterile saline solution. Preferably, upon reconstitution, the antibody contained therein is stable, soluble and demonstrates tolerability upon administration to a patient.

The pharmaceutical compositions of the present invention are characterized by their tolerability. As used herein, the terms “tolerable” and “tolerability” refer to the ability of the pharmaceutical compositions of the present invention to not elicit an adverse reaction in the patient to whom such composition is administered, or alternatively not to elicit a serious adverse reaction in the patient to whom such composition is administered. In some embodiments, the pharmaceutical compositions of the present invention are well tolerated by the patient to whom such compositions is administered.

Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition that comprises the antibody disclosed herein may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Some embodiments include at least one of the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The formulations to be used for in vivo administration (e.g., SC administration) may be sterile. In some embodiments, the formulation is sterilized by filtration through sterile filtration membranes.

In some embodiments, the anti-CD19 antibodies (e.g., obexelimab) disclosed herein are formulated for SC administration. In some embodiments, the anti-CD19 antibody (e.g., obexelimab) formulation for SC administration comprises one or more buffers, one or more tonicity modifiers, one or more solvents, and one or more surfactants. Nonlimiting examples of buffers include phosphate, citrate, acetate, glutamate, carbonate, tartrate, triethanolamine (TRIS), glycylglycine, histidine, glycine, lysine, arginine, and other organic acids. More specifically, non-limiting examples of buffers include HEPES sodium, MES, potassium phosphate, potassium thiocyanate, sterilant, TAE, TBE, ammonium sulfate/HEPES, BuffAR, sodium acetate, sodium carbonate, sodium citrate, sodium dihydrogen phosphate, disodium hydrogen phosphate, and sodium phosphate. Additionally, the buffer may be various hydrate forms. For example, the buffer may be a monohydrate, a dihydrate, a trihydrate, a tetrahydate, a pentahydrate, a hexahydrate, a heptahydrate, an octahydrate, a nonahydrate, a decahydrate, an undecahydrate, and a dodecahydrate. Occasionally, a hydrate may be fractional such as a hemihydrate or a sequihydrate. Nonlimiting examples of tonicity modifies include sodium chloride, acetic acid, L-proline, dextrose, mannitol, potassium chloride, glycerin, and glycerol. Non-limiting example of solvents include water, propylene glycol, polyethylene glycols, ethanol, dimethyl sulfoxide, N-methyl-2-pyrrolidone, glycofurol, Solketal™, glycerol formal, acetone, tetrahydrofurfuryl alcohol, diglyme, dimethyl isosorbide, and ethyl lactate. Non-limiting examples of solvents include polysorbates (e.g. polysorbate-20, polysorbate-80), polyoxyethylene sorbitan monooleate (Tween 80), sorbitan monooleate polyoxyethylene sorbitan monolaurate (Tween 20), sorbitan trioleate (span 85), lecithin, and polyoxyethylene polyoxypropylene copolymers (Pluronics, Pluronic F-68).

The amounts of anti-CD19 antibody (e.g., obexelimab), buffer, tonicity modifier, solvent, and surfactants may vary. In some embodiments, the anti-CD19 antibody (e.g., obexelimab) is formulated at a concentration of 125 mg/mL obexelimab, 2.35 mg/mL sodium acetate trihydrate, 0.17 mg/mL acetic acid (at density 1.053 g/mL), 30 mg/mL L-proline, 0.1 mg/mL polysorbate 80, pH 5.5. In some embodiments, the anti-CD19 antibody (e.g., obexelimab) is formulated at a concentration of 80-200 mg/mL obexelimab, 1.5-3 mg/mL sodium acetate trihydrate, 0.1-0.2 mg/mL acetic acid (at density 1.053 g/mL), 10-50 mg/mL L-proline, 0.05-0.2 mg/mL polysorbate 80, pH 5.0-6.0. In some embodiments, the anti-CD19 antibody (e.g., obexelimab) is formulated at a concentration of 122-127 mg/mL obexelimab, 2.0-2.5 mg/mL sodium acetate trihydrate, 0.15-0.19 mg/mL acetic acid (at density 1.053 g/mL), 25-35 mg/mL L-proline, 0.05-0.15 mg/mL polysorbate 80, pH 5.0-6.0.

In certain embodiments, a SC formulation comprises the anti-CD19 antibody (e.g., obexelimab), one or more buffers, one or more tonicity modifiers, one or more solvents, and one or more surfactants. In some embodiments, the buffer can be a sodium acetate buffer. For example, the buffer can be sodium acetate trihydrate. In an embodiment, the tonicity modifier can be acetic acid, L-proline, and combinations thereof. In another embodiment, the solvent is water.

In some embodiments, the surfactant is a polysorbate. In some embodiments, the polysorbate is polysorbate-80. In some embodiments, a SC formulation comprises the anti-CD19 antibody (e.g., obexelimab), sodium acetate trihydrate, acetic acid and L-proline, water, and polysorbate-80.

In some embodiments, a SC formulation comprises the anti-CD19 antibody (e.g., obexelimab) in an amount from 1 mg to 500 mg per mL or 50 mg to 250 mg per mL or 100 mg to 250 mg per mL, sodium acetate trihydrate in an amount from 1 to 1 0 mg per mL or 1 to 5 mg per mL or 1 to 2.5 mg per mL, acetic acid and L-proline in an amount from 5 to 50 mg per mL or 10 to 50 mg per mL or 20 to 40 mg per mL, water up to about 1 mL, and polysorbate-80 in an amount from 0.01 mg to 1 mg per mL or 0.01 to 0.5 mg/mL or 0.05 to 0.2 mg/mL. Specifically, a SC formulation comprises the anti-CD19 antibody (e.g., obexelimab) in an amount from 100 to 250 mg/mL, sodium acetate trihydrate in an amount from 1 to 2.5 mg/mL, acetic acid and L-proline in an amount from 20 to 40 mg/mL, water up to 1 mg/mL, and polysorbate-80 in an amount from 0.05 to 0.2 mg per mL.

In some embodiments, a SC formulation comprises obexelimab, a buffer, and a tonicity modifier. In some embodiments, a SC formulation comprises 100 mg/mL to 250 mg/mL obexelimab, an acetate buffer, and proline. In some embodiments, a SC formulation comprises 125 mg/mL obexelimab, an acetate buffer, and proline. In some embodiments, a SC formulation comprises 100 mg/mL to 250 mg/mL obexelimab, 5 to 40 mM acetate buffer, and 1% to 5% (w/v) proline. In some embodiments, a SC formulation comprises 125 mg/mL obexelimab, 20 mM acetate buffer, and 3% (w/v) proline, at pH 5 to 6. In some embodiments, a SC formulation comprises 125 mg/mL obexelimab, 20 mM acetate buffer, and 3% (w/v) proline at pH 5.5.

In some embodiments, a SC formulation comprises obexelimab, a buffer, a tonicity modifier, and a surfactant.

In some embodiments, a SC formulation comprises 100 mg/mL to 250 mg/mL obexelimab, an acetate buffer, proline, and polysorbate 80.

In some embodiments, a SC formulation comprises 100 mg/mL to 250 mg/mL obexelimab, 5 to 40 mM acetate buffer, 1% to 5% (w/v) proline, and 0.002% to 0.02% (w/v) polysorbate 80.

In some embodiments, a SC formulation comprises 125 mg/mL obexelimab, 20 mM acetate buffer, 3% (w/v) proline, and 0.01% (w/v) polysorbate 80, at pH 5 to 6.

In some embodiments, a SC formulation comprises 125 mg/mL obexelimab, 20 mM acetate buffer, 3% (w/v) proline, and 0.01% (w/v) polysorbate 80 at pH of 5.5.

In some embodiments, a SC formulation comprises 125 mg/mL obexelimab, 2.35 mg/mL sodium acetate trihydrate, 0.17 mg/mL acetic acid (at density 1.053 g/mL), 30 mg/mL L-proline, and 0.1 mg/mL polysorbate 80. In some embodiments, a SC formulation has a pH of 5.5.

In some embodiments, a SC formulation has a unit dose strength(s)/dosage level(s) that is 125.0 (±10%) mg/mL of obexelimab.

Anti-CD19 antibodies (e.g., obexelimab) as disclosed herein may also be formulated as immunoliposomes. A liposome is a small vesicle comprising various types of lipids, phospholipids and/or surfactant that is useful for delivery of an anti-CD19 antibody (e.g., obexelimab) to a mammal. Liposomes containing the anti-CD19 antibody (e.g., obexelimab) are prepared by methods known in the art. Liposomes with enhanced circulation time are disclosed in U.S. Pat. No. 5,013,556, incorporated entirely by reference. In some embodiments, the anti-CD19 antibody (e.g., obexelimab) is formulated in liposomes generated by the reverse phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

In some embodiments, the anti-CD19 antibody (e.g., obexelimab) is entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nanoparticles and nanocapsules), and macroemulsions. Such techniques are disclosed in Remington's Pharmaceutical Sciences 16^{th} edition, Osol, A. Ed., 1980, incorporated entirely by reference.

In some embodiments, sustained-release preparations may be prepared to deliver the anti-CD19 antibody (e.g., obexelimab). Suitable examples of sustained-release preparations include semipermeable matrices of solid hydrophobic polymer, which matrices are in the form of shaped articles, e.g., films, or microcapsules. Examples of sustained-release matrices include polyesters, hydrogels (for example poly(2-hydroxyethyl-methacrylate), or poly(vinylalcohol)), polylactides (U.S. Pat. No. 3,773,919, incorporated entirely by reference), copolymers of L-glutamic acid and gamma ethyl-L-glutamate, non-degradable ethylene-vinyl acetate, degradable lactic acid-glycolic acid copolymers such as the Lupron Depot® (which are injectable microspheres composed of lactic acid-glycolic acid copolymer and leuprolide acetate), poly-D-(-)-3-hydroxybutyric acid, and ProLease® (commercially available from Alkermes), which is a microsphere-based delivery system composed of the desired bioactive molecule incorporated into a matrix of poly-DL-lactide-co-glycolide (PLG).

### Contains for Injection

The present invention provides containers for injecting pharmaceutical compositions and formulations of anti-CD19 antibodies (e.g., obexelimab). In one aspect, a container comprises a liquid pharmaceutical composition comprising obexelimab. Suitable containers include, without limitation, a syringe, an autoinjector, vial, infusion bottle, ampoule, carpoule, a syringe equipped with a needle protection system, and a carpoule within an injection pen.

In some embodiments, a container comprising the liquid pharmaceutical composition is a prefilled syringe, a vial, or an autoinjector. In some embodiments, a container is a prefilled syringe. In some embodiments a container is an autoinjector. In some embodiments, an autoinjector administers a 2.0 mL fixed injection dose volume. In some embodiments, an autoinjector administers a 2.0 mL fixed injection dose volume using a 27-gauge needle. In some embodiments, an autoinjector is suitable for once weekly administration of an effective amount of the obexelimab composition to a patient (e.g., any formulation described herein).

In some embodiments, a container is a prefilled syringe or autoinjector comprising a liquid pharmaceutical composition comprising:
a. 122-127 mg/mL obexelimab,
b. 2.0-2.5 mg/mL sodium acetate trihydrate, at a pH 5.0-6.0,
c. 0.15-0.19 mg/mL acetic acid (at density 1.053 g/mL),
d. 25-35 mg/mL L-proline,
e. 0.05-0.15 mg/mL polysorbate 80.

In some embodiments, a prefilled syringe or autoinjector comprises a liquid pharmaceutical composition comprising: a liquid pharmaceutical composition comprises 125 mg/mL obexelimab, 2.35 mg/mL sodium acetate trihydrate, 0.17 mg/mL acetic acid, 30 mg/mL L-proline, 0.1 mg/mL polysorbate 80 at pH 5.5.

In some embodiments, a prefilled syringe or autoinjector comprises a liquid pharmaceutical composition having a unit dose strength of 125.0 (±10%) mg/mL of obexelimab.

In some embodiments, a prefilled syringe or autoinjector comprises a liquid pharmaceutical composition comprising: 125 mg/mL obexelimab, 20 mM acetate buffer, 3% (w/v) proline, 0.01% (w/v) polysorbate 80, at pH 5.5.

In some embodiments, a prefilled syringe or autoinjector facilitates SC or intradermal delivery of the pharmaceutical composition. In some embodiments, a method of treating RMS described herein comprises, administering a formulation comprising obexelimab into the patient's bloodstream following a single or multiple SC injection to the abdomen of the patient using the prefilled syringe or autoinjector.

In some embodiments, a method of treating RMS comprises administering a single weekly injection of a 2.0 mL fixed injection dose. In some embodiments, a method of treating RMS comprises administering a single weekly injection of a 2.0 mL of 125 mg/mL obexelimab, 20 mM acetate buffer, 3% (w/v) proline, 0.01% (w/v) polysorbate 80, at pH 5.5.

In some embodiments, a composition in the prefilled syringe is stable for at least 3 months when stored at 2-8° C. In some embodiments, a composition in a pre-filled syringe is stable for at least 6 months when stored at 2-8° C. In some embodiments, a composition in a pre-filled syringe is stable for at least 6 months when stored at 2-8° C. In some embodiments, a composition in a pre-filled syringe is stable for at least 1 year when stored at 2-8° C. In some embodiments, a composition in a pre-filled syringe is stable for at least 2 years when stored at 2-8° C.

### EXAMPLES

While certain methods of the present invention have been described with specificity in accordance with certain embodiments, the following examples serve only to illustrate the methods of the invention and are not intended to limit the same.

### Example 1. A Phase 2, Multicenter, Randomized, Double-Blind, Placebo-Controlled Study

This study will evaluate the efficacy and safety of obexelimab administered SC once per week (QW) in patients with RMS. MS is a chronic, immune-mediated neurological disorder characterized by inflammation, demyelination, and axonal damage in the central nervous system with a wide variety of clinical phenotypes (Dimitriou 2023). The core MS phenotypes are RRMS disease and progressive disease. The disease subset of interest of RMS is characterized by a clinical course with defined recurrent attacks of new or exacerbated neurological dysfunction. Disease modifying therapies for MS patients significantly decrease morbidity by decreasing frequency of relapses and slowing down the progression of the disease.

### Study Design:

This is a Phase 2, multicenter, double-blind, placebo-controlled study to evaluate the efficacy and safety of obexelimab in patients with RMS. The study will consist of a Screening Period (Day-28 to Day-1), followed by a 76-week Treatment Period comprising Part A (12 weeks of randomized, placebo controlled treatment), Part B (12 weeks of open-label treatment with obexelimab), and Part C (52 weeks of open-label treatment with obexelimab followed by a 12-week post-treatment safety Follow-up Period). Including screening and follow-up, the expected maximum duration of participation in this study for an individual patient is approximately 92 weeks (i.e. 28-day Screening Period, 24-week Treatment Period [Parts A and B], 52-week Open Label Extension [Part C], and a 12-week post-treatment safety Follow-up Period).

The SC formulation of obexelimab is a sterile liquid product supplied as a single-use, glass prefilled syringe with a half-inch 27-gauge staked needle containing 1 mL (125 mg) of obexelimab investigation medicinal product (IMP). Two injections of 1 mL each will be administered SC. Qualified site staff will administer the first injection of IMP on Day 1 (Week 0) and will instruct the patient/designated caregiver on how to properly self-administer/administer IMP. For the second injection (to complete the first dose administration on Day 1), patients/designated caregivers capable of self-administration/administration may self-administer/administer IMP under the supervision of site staff.

### Part A

Following signing of the informed consent form (ICF), all screening procedures will be performed during the Screening Period within 28 days prior to randomization. Patients enrolled in the study will have a diagnosis of RMS according to the 2017 revision of the McDonald diagnostic criteria, an Expanded Disability Status Scale (EDSS) of ≤5.5 at the Screening Visit, and documentation of one of the following:
at least 1 relapse within the previous year
or ≥2 relapses within 2 previous years
or ≥1 active GdE brain lesion on an MRI scan within the past 6 months prior to screening.

Screening MRI data will be adjudicated by a central Adjudication Committee (AC) to confirm the appropriate MS diagnosis prior to randomization.

On Study Day 1, patients will be randomized 2:1 to receive obexelimab 250 mg or placebo SC QW for 12 weeks. Each weekly dose will comprise two 1-mL (125 mg/mL) injections. Randomization will be stratified based on disease activity (Gd lesion status of ≥1 versus 0 at screening). To explore the potential impact on pharmacokinetics (PK) and pharmacodynamics (PD) in the CSF of patients with MS, additional consent will be obtained in approximately 10% of enrolled patients for optional CSF assessment via lumbar puncture.

### Part B

At the Week 12 visit, all patients will receive open-label obexelimab SC QW for an additional 12 weeks.

### Part C

At Week 24, patients will proceed to Part C for an additional 64 weeks (a 52-week treatment open-label period followed by a 12-week post-treatment safety Follow-up Period).

At the Week 24 visit, the study will continue as an open-label extension for an additional 52 weeks. Administration of obexelimab will occur at home every 7 days (±2 days) between in-clinic visits. All doses of IMP will be self-administered unless the patient needs additional assistance, in which case patients may utilize a designated patient caregiver for IMP administration.

### Patient Population

To be eligible for the study, the following criteria apply:
1. Capable of giving signed informed consent, which includes compliance with the requirements and restrictions listed in the ICF
2. Male or female, ≥18 to ≤60 years of age, inclusive, at the time of signing the ICF
3. Diagnosis of RMS (relapsing-remitting or active secondary progressive) according to the 2017 revision of the McDonald diagnostic criteria
4. An EDSS of ≤5.5 at the Screening Visit
5. Must have documentation of:
   a. at least 1 relapse within the previous year OR
   b. ≥2 relapses within the past 2 years OR
   c. ≥1 active Gd-enhancing brain lesion on an MRI scan within the past 6 months prior to screening.

### Exclusion Criteria

Eligibility does not apply if these criteria apply:
a) Primary progressive MS or inactive secondary progressive MS
b) Meet criteria for neuromyelitis optica spectrum disorder
c) Relapse in the 30 days prior to randomization
d) ≥10 years disease duration from onset with patient's EDSS ≤2.0 (patient reported is adequate in absence of written medical record)
e) Has >20 Gd+ lesions on brain MRI at screening

### Objectives

The primary objective of the study is to evaluate the effect of weekly SC administration of obexelimab versus placebo in patients with RMS on the prevention of new GdE T1 hyperintense lesions detected using MRI. Secondary objectives of the study include evaluating the effect of weekly SC administration of obexelimab versus placebo in patients with RMS on additional MRI endpoints and neurofilament light chain. Further objectives include evaluating the safety and tolerability, PK profile and anti-drug antibodies (ADA), and PD parameters of weekly SC administration of obexelimab in patients with RMS. Exploratory endpoints include evaluating the effect of weekly SC administration of obexelimab in patients with RMS over 24 weeks as noted below.

### Disease Activity Assessment:

Patient-reported outcome (PRO) assessments will be conducted prior to all other assessments and procedures. PROs will include assessing Patient Reported Outcome Measuring System (PROMIS)-Fatigue-MS-8, Patient Global Impression of Severity (PGIS)-Fatigue, Patient Global Impression of Change (PGIS)-Fatigue, and Multiple Sclerosis Impact Scale-29.

Disease activity will be monitored in part through assessment of newly developed lesions and volume of prespecified lesions via MRI. Brain MRI (both before and after administration of Gd contrast agent) will be performed. The basic MRI will be performed at all sites and will consist of T2- and T1-weighted sequences before and after administration of the contrast agent.

An expanded protocol will be conducted using susceptibility-weighted imaging centers with capacity of 3T MRI. Traditional MRI sequences will be used to evaluate MRI-related endpoints such as those mentioned in the study endpoints section. As the use of corticosteroids can affect the MRI outcomes, patients receiving corticosteroids for MS relapse must have a 3-week interval between the last dose of corticosteroids and the scheduled MRI scan.

Patients will further be assessed by a clinician performing a standard neurological examination. Disability will be assessed using the EDSS. The EDSS-assessing clinician will be blind to all MRI results.

### Pharmacokinetics:

PK samples (blood and CSF) will be collected from patients (i.e. intermittent sampling). Drug concentration will be determined using a validated method.
Obexelimab serum concentrations from baseline through Week 24
Obexelimab CSF concentrations from baseline and at Weeks 12 and 24
Incidence of anti-obexelimab antibodies through Week 36

### Pharmacodynamics:

Samples for biomarker evaluations (blood and CSF) will be collected according to the schedule of assessments. Collection of all PD/biomarker samples must be performed prior to IMP administration. Venous blood samples and CSF samples via LP will be collected for biomarker measurements. PD biomarkers being evaluated in this study may include:
Gene expression immune pathway modules (e.g. B cell module) in whole blood
Cytokines/chemokines and neurology markers (e.g. NfL, Tau, GFAP, CHI3L1, CXCL-13, UCH-L1, and TREM2) in serum
Circulating immune cells (e.g. T, B, and natural killer cells) in whole blood
CD19 target receptor occupancy (samples drawn and tested at study sites in North America only) in whole blood
Immunoglobulin levels (e.g. IgG, IgA, IgM) in serum

Values and changes from baseline at Weeks 12 and 24 in CSF:
CSF immunoglobulin levels (e.g. oligoclonal IgG bands, IgA, IgM)
CSF cytokines/chemokines and neurology markers (e.g. NfL, Tau, GFAP, CHI3L1, CXCL-13, UCH-L1, and TREM2)
CSF immune cells (e.g. B cells, T cells phenotyping and/or gene expression)

### Endpoints:

### Primary

Cumulative number of new GdE T1 hyperintense lesions over Week 8 and Week 12, as measured by brain MRI

### Secondary

Cumulative number of new and/or enlarging T2 weighted hyperintense lesions detected over Week 8 and Week 12
Number of new GdE T1 hyperintense lesions at Week 4, Week 8, and Week 12
Change from baseline in volume of T2 lesions at Week 12
Serum NfL at Week 12

### Exploratory

### Efficacy

Number of new GdE T1 hyperintense lesions at Week 24
Number of new and/or enlarging T2 lesions at Week 24, Week 48, and Week 76
Change from baseline in volume of T2 lesions at Week 24, Week 48, and Week 76
Number of GdE T1 hyperintense lesions at 12 weeks, 48 weeks, and 76 weeks of obexelimab treatment
Total number of GdE T1 hyperintense lesions at Week 24 in comparison with Week 12
Number of new T1-hypointense lesions at Week 12, Week 24, Week 48, and Week 76
Proportion of patients with no new MRI disease activity at Week 12, Week 24, Week 48, and Week 76
New phase rim lesions arising from acute lesions at Week 4, Week 8, Week 12, Week 24, Week 48, and Week 76 (selected centers)
Number of phase rim lesions (selected centers) at Week 12, Week 24, Week 48, and Week 76
Change in quantitative values within lesions and surrounding tissue from baseline to Week 12, Week 24, Week 48, and Week 76
Change in brain volume, including regional changes but not limited to thalamus and cerebral cortex, from baseline to Weeks 12, and 24
Brainstem upper cervical SC 3D conformational measures (selected centers) from baseline to Week 12, Week 24, Week 48, and Week 76
Change from baseline in number, volume, and intensity (T1) of slowly expanding lesions (SELs) at Week 24, Week 48, and Week 76
Number of relapses (ARR) over time from baseline to Week 12, Week 24, Week 48 and Week 76
Proportion of relapse-free patients over time from baseline to Week 12, Week 24, Week 48, and Week 76
Change in EDSS from baseline to Week 12, Week 24, Week 48, and Week 76
Proportion of patients with 3 month and/or 6 month Confirmed Disability Progression confirmed at Weeks 12 and 24
Change in PROMIS-Fatigue-MS-8 scoring from baseline to Week 12, Week 24, Week 48, and Week 76
Change in PGIS-Fatigue from baseline to Week 12, Week 24, Week 48, and Week 76
PGIC-Fatigue value at Week 12, Week 24, Week 48, and Week 76
Change in MSIS-29 physical and psychological domains scoring from baseline to Week 12, Week 24, Week 48, and Week 76
Relative change from baseline in serum NfL from baseline to Week 12

### PK and ADA

Obexelimab serum concentrations at Weeks 24, 36, 48, 60, and 76
Obexelimab CSF concentrations at Week 12, Week 24, and Week 76
Incidence of anti-obexelimab antibodies through Week 20

### PD

Values and changes from baseline through Week 24 and Week 76 in blood:
   Immune pathway modules (e.g., B cell modules)
   Cytokines/chemokines and neurology markers (e.g., NfL, Tau, GFAP, CHI3L1, CXCL-13, UCH-L1, and TREM2)
   Circulating immune cells
   Immunoglobulin levels (e.g., IgG, IgA, IgM)
Values and changes from baseline through Week 12, Week 24, and Week 76 in CSF:
   Cytokines/chemokines and neurology markers (e.g., NfL, Tau, GFAP, CHI3L1, CXCL-13, UCH-L1, and TREM2)
   Immune cells (e.g., B cells, T cells phenotyping, and/or gene expression)
   Ig levels (e.g., oligoclonal IgG bands, IgA, IgM)

1. A method of treating relapsing forms of multiple sclerosis (RMS) comprising:
administering to a patient in need of treatment an anti-CD19 antibody at a therapeutically effective dosing regimen,
wherein the anti-CD19 antibody comprises a light chain and a heavy chain,
wherein the light chain comprises a LCDR1 of SEQ ID NO:2, a LCDR2 of SEQ ID NO:3, and a LCDR3 of SEQ ID NO:4,
wherein the heavy chain comprises a HCDR1 of SEQ ID NO:5, a HCDR2 of SEQ ID NO:6, and a HCDR3 of SEQ ID NO:7, and
wherein the heavy chain comprises an Fc region comprising amino acid substitutions S267E and L328F wherein numbering is according to the EU index.

2. A method of treating primary-progressive multiple sclerosis (PPMS) comprising:
administering to a patient in need of treatment an anti-CD19 antibody at a dose of 250 mg every week, or an equivalent dosing regimen thereof,
wherein the anti-CD19 antibody comprises a light chain and a heavy chain,
wherein the light chain comprises a LCDR1 of SEQ ID NO:2, a LCDR2 of SEQ ID NO:3, and a LCDR3 of SEQ ID NO:4,
wherein the heavy chain comprises a HCDR1 of SEQ ID NO:5, a HCDR2 of SEQ ID NO:6, and a HCDR3 of SEQ ID NO:7, and
wherein the heavy chain comprises an Fc region comprising amino acid substitutions S267E and L328F wherein numbering is according to the EU index.

3. The method of claim 1 or 2, wherein the therapeutically effective dosing regimen comprises administering the anti-CD19 antibody at a dose of 250 mg every week, or an equivalent dosing regimen thereof.

4. A method of treating multiple sclerosis (MS) comprising:
administering to a patient in need of treatment an anti-CD19 antibody at a dose of 250 mg every week, or an equivalent dosing regimen thereof,
wherein the anti-CD19 antibody comprises a light chain and a heavy chain,
wherein the light chain comprises a LCDR1 of SEQ ID NO:2, a LCDR2 of SEQ ID NO:3, and a LCDR3 of SEQ ID NO:4,
wherein the heavy chain comprises a HCDR1 of SEQ ID NO:5, a HCDR2 of SEQ ID NO:6, and a HCDR3 of SEQ ID NO:7, and
wherein the heavy chain comprises an Fc region comprising amino acid substitutions S267E and L328F wherein numbering is according to the EU index.

5. The method of any one of claims 1 and 3-4, wherein the patient has relapsing-remitting MS (RRMS), secondary progressive MS (SPMS), or clinically isolated syndrome (CIS).

6. The method of claim 5, wherein the patient has RRMS.

7. The method of claim 5, wherein the patient has SPMS.

8. The method of claim 5, wherein the patient has CIS.

9. The method of any one of the preceding claims, wherein the patient has an Expanded Disability Status Scale (EDSS) score of ≤5.5 prior to commencing treatment.

10. The method of any one of the preceding claims, wherein the patient has an EDSS score of ≥5.0 prior to commencing treatment.

11. The method of any one of the preceding claims, wherein the patient has experienced at least one relapse within the previous year, or at least two relapses within the previous two years prior to commencing treatment.

12. The method of any one of the preceding claims, wherein the administration of the anti-CD19 antibody is sufficient to stabilize or reduce EDDS score.

13. The method of any one of the preceding claims, wherein the administration of the anti-CD19 antibody reduces the level of neurofilament light chain (NfL) in the patient's blood as compared to pre-treatment level.

14. The method of any one of the preceding claims, wherein the patient has developed at least one active gadolinium-enhancing (GdE) brain lesion on an MRI scan within the past 6 months prior to commencing treatment.

15. The method of any one of the preceding claims, wherein the administration of the anti-CD19 antibody stabilizes or reduces GdE T1 hyperintense lesions as compared to pre-treatment status.

16. The method of any one of the preceding claims, wherein the administration of the anti-CD19 antibody stabilizes or reduces GdE T2 hyperintense lesions as compared to pre-treatment status.

17. The method of any one of the preceding claims, wherein the anti-CD19 antibody is administered for at least 8 weeks, at least 12 weeks, at least 24 weeks, or at least 76 weeks.

18. The method of any one of the preceding claims, wherein the light chain comprises a light chain variable region that is at least 90% identical to SEQ ID NO:11, and wherein the heavy chain comprises a heavy chain variable region that is at least 90% identical to SEQ ID NO:12.

19. The method of claim 18, wherein the heavy chain variable region comprises SEQ ID NO:12; and the light chain variable region comprises SEQ ID NO:11.

20. The method of any one of the preceding claims, wherein the heavy chain comprises an amino acid sequence at least 90% identical to SEQ ID NO:10, and the light chain comprises an amino acid sequence at least 90% identical to SEQ ID NO:9.

21. The method of any one of the preceding claims, wherein the heavy chain comprises SEQ ID NO:10, and a light chain comprises SEQ ID NO:9.

22. The method of any one of the preceding claims, wherein the anti-CD19 antibody is administered subcutaneously.

23. The method of any one of the preceding claims, wherein the anti-CD19 antibody is administered in a liquid formulation comprising 125 mg/mL anti-CD19 antibody.

24. The method of claim 23, wherein the liquid formulation further comprises 2.35 mg/mL sodium acetate trihydrate, 0.17 mg/mL acetic acid, 30 mg/mL L-proline, 0.1 mg/mL polysorbate 80 at pH 5.5.

25. The method of any one of claims 22-24, wherein the anti-CD19 antibody is administered using a prefilled syringe or autoinjector.


SEQUENCE LISTING (12 sequences)

SEQ ID NO: 1 - protein, 556 aa, Homo sapiens [sequence omitted]

SEQ ID NO: 2 - protein, 16 aa, synthetic construct
RSSKSLQNVNGNTYLY

SEQ ID NO: 3 - protein, 7 aa, synthetic construct
RMSNLNS

SEQ ID NO: 4 - protein, 9 aa, synthetic construct
MQHLEYPIT

SEQ ID NO: 5 - protein, 5 aa, synthetic construct
SYVMH

SEQ ID NO: 6 - protein, 14 aa, synthetic construct
WIGYINPYNDGTKY

SEQ ID NO: 7 - protein, 12 aa, synthetic construct
GTYYYGTRVFDY

SEQ ID NO: 8 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 12 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS